(19) United States
(12) Patent Application Publication
(10) Pub. No.: US 2022/0056528 A1
(43) Pub. Date: Feb. 24, 2022

(54) METHOD OF DETECTING INFECTION WITH PATHOGENS CAUSING TUBERCULOSIS

(71) Applicant: MIKROGEN GMBH, Neuried (DE)

(72) Inventors: Hilmar DEML, Regenstauf (DE); Anne RASCLE, Penting (DE); Sascha BARABAS, Bad Abbach (DE); Alexandra ASBACH-NITZSCHE, Regensburg (DE)

(21) Appl. No.: 17/416,207

(22) PCT Filed: Dec. 20, 2019
(86) PCT No.: PCT/EP2019/086579

§ 371 Date: Jun. 18, 2021

(30) Foreign Application Priority Data
Dec. 20, 2018 (EP) 18214607.6

(51) Int. Cl. C12Q 1/6883

(52) U.S. Cl. CPC C12Q 1/6883; C12Q 2600/158

(57) ABSTRACT

The present invention refers to in vitro methods of detecting an infection with pathogens causing tuberculosis comprising the steps of (a) contacting a first aliquot of a sample of an individual with at least one antigen of a pathogen causing tuberculosis, b) incubating the first aliquot with the at least one antigen over a certain period of time, c) detecting in the first aliquot and in a second aliquot of the sample of the individual a marker or a combination of markers, e.g. Interferon gamma, CXCL10, ncTRIM69, using reverse transcription quantitative real-time polymerase chain reaction (RT-qPCR) or RNA Sequencing (RNA-Seq), and d) comparing the detected marker(s) in the first aliquot with the detected marker(s) in the second aliquot, wherein the second aliquot has not been incubated with the at least one antigen. In addition, the present invention refers to a kit for performing the methods according to the present invention. The present invention also refers to the use of the marker ncTRIM69, a primer for amplification of the marker ncTRIM69, and/or a probe for detecting the marker ncTRIM69 in an in vitro method of diagnosing tuberculosis, in particular of detecting infection with pathogens causing tuberculosis.

The present invention refers to in vitro methods of detecting an infection with pathogens causing tuberculosis comprising the steps of (a) contacting a first aliquot of a sample of an individual with at least one antigen of a pathogen causing tuberculosis, b) incubating the first aliquot with the at least one antigen over a certain period of time, c) detecting in the first aliquot and in a second aliquot of the sample of the individual a marker using reverse transcription quantitative real-time polymerase chain reaction (RT-qPCR) or RNA Sequencing (RNA-Seq), and d) comparing the detected marker(s) in the first aliquot with the detected marker(s) in the second aliquot, wherein the second aliquod has not been incubated with the at least one antigen. In addition, the present invention refers to a kit for performing the methods according to the present invention. The present invention also refers to the use of the marker ncTRIM69, a primer for amplification of the marker ncTRIM69, and/or a probe for detecting the marker ncTRIM69 in an in vitro method of diagnosing tuberculosis, in particular of detecting infection with pathogens causing tuberculosis.

Tuberculosis is a widespread infectious disease, which is caused by different strains of mycobacteria (in particular Mycobacterium tuberculosis, Mtb). It affects primarily the lung (pulmonary TB) with manifestations in other areas of the body such as lymph nodes, urinary tract, bones, joints and the gastrointestinal tract (extrapulmonary TB). According to estimates of the world health organisation (WHO) in 2014 approximately 1.7 million people died from tuberculosis. Thus tuberculosis remains one of the three major deadly infectious diseases worldwide. In addition worldwide approximately two billion humans are latently infected with the pathogen and the number increases by approximately 10.4 million new cases per year (WHO Global Tuberculosis Report 2017).

During lifetime, approximately, 10-15% of the latently infected immunocompetent individuals develop a treatment requiring active tuberculosis. Substantially higher numbers of reactivations are observed in patients with impaired immune function such as HIV patients.

Considering the lack of an effective, broadly protective vaccine, a rapid and reliable diagnosis of mycobacterial infection remains an important step to identify infected individuals and thus to perform differential diagnosis of the status of disease and to initiate appropriate, personalized treatment.

The currently available methods for the diagnosis of mycobacterial infections can be classified in three groups:
patient anamnesis and clinical symptoms
methods for direct pathogen detection
methods for the detection of mycobacteria-specific cellular immune reactions

Besides patient anamnesis, X-ray examination and bacterial diagnostics remain centrial clinical methods for a comprehensive diagnosis of the status of tuberculosis.

X-ray examination: Till today, X-ray examination plays an important role in the detection of active tuberculosis and monitoring of therapy success. Beyond that this method provides important directions regarding the early diagnosis as well as the exclusion of treatment requiring tuberculosis at tuberculin skin test (TST) and/or interferon-gamma release (IGRA)-test positive contact persons. Advantages of these methods are the high sensitivity, however with reduced specificity.

Microscopy: Sputum microscopy allows a rapid evaluation of the infectivity of a patient on suspicion for pulmonary tuberculosis. Limitations of the method are the low sensitivity of 50 to 70%. In addition, the assay allows no discrimination between living and dead bacteria and no species allocation.

Culture: Direct detection of the pathogen by culture represents the gold standard for the diagnosis of an active tuberculosis with high sensitivity and specificity. However, the method suffers from the long time to result (available at least after 2 to 4 weeks).

Nucleic amplification tests (NAT): NAT such as the GeneXpert MTB/RIF test (Cepheid Inc., Sunnyvale, USA) are primarily used for indication examinations to confirm reasonable suspicion for tuberculosis in sputum-negative patients. In addition, NAT enables a rapid discrimination of mycobacteria from non-tuberculous mycobacteria in patients with microscopy-positive sputum. However, these tests show limitations in patients with low bacterial load and patients suffering from extrapulmonary tuberculosis; latter represent at least 15 to 20% of all tuberculosis cases. In addition, the test is not suitable for children, as for children the extraction of sputum (by coughing from the depth of the lung) is very difficult and painful. In addition, NAT are not suitable for the control of therapy success as these tests also detect DNA or RNA of non-viable bacteria.

Immunological methods: Besides methods for the direct detection of pathogens particularly in industrialized countries immunological detection methods gain increasing importance. These tests are based on the detection of Mtb polypeptide-specific immune reactions as indirect “host-response” marker for an infection with a mycobacterial pathogen. The most prominent representative is the tuberculin scin test (TST), which has already been applied as a diagnostic test for more than one century. This method is characterized by a high sensitivity but a limited specificity. For example cross reactivity with nontuberculous mycobacteria or a vaccination with nontuberculous mycobacteria or vaccination with the BCG (Bacille Calmette-Guerin)-vaccine strain can lead to false positive test results. Otherwise, TST results can be false negative in immunocompromized patients such as HIV patients or transplant patients treated with immunosuppressive substances. In addition, false negative test results can arise during the pre-allergic phase of infection and at severe courses of a general disease. Thus, a negative TST result does not exclude the presence of tuberculosis.

In contrast to TST the since 2005 commercially available interferon-gamma release tests (IGRAs) allow for the first time a differentiation of infected patients from vaccinated individuals. The test bases on the specific detection of M. tuberculosis polypeptide-reactive memory T cells, which are generated within the course of a mycobacterial infection. Renewed contact with M. tuberculosis polypeptides results in a specific reactivation of these cells coinciding with the production of characteristical marker cytokines.

The IGRA tests are based on the stimulation of isolated blood cells or anticoagulated whole blood of a patient with preselected Mtb polypeptides and the subsequent determination of the number of marker cytokine (mostly IFN-γ)-producing cells (T-Spot-TB test, (Oxford Immunotec Ltd., Oxford UK)) or the quantification of produced marker cytokine (e.g. IFN-γ) by ELISA (Quantiferon-TB Gold in Tube (QFT-GIT), Qiagen, Hilden, Germany). Herein, the numbers of cytokine secreting cells or the concentrations of specifically secreted marker cytokines serve as an indirect immunological marker for the detection of mycobacterial infection.

Compared to the TST test the IGRA assays show subsequently described advantages: no significant distorsion of the test result by BCG vaccination or infection with almost all non-tuberculous mycobacteria (NTM). In addition, in contrast to the TST test performance of the in vitro IGRA assay does not stimulate of patient's immune system and thus to a falsification of subsequent measurements; in addition there is no need for a second visit to perform the assay.

One important limitation of both types of IGRA assays is the not satisfactory sensitivity and specificity, whereby widely disparate test results have been reported in different studies. A meta-analysis based on the evaluation of 157 studies published in 2017 by Doan and coworkers reported test sensitivities for the TST, QFT-GIT and the T-Spot-TB test in immunocompetent adults for the detection of latent tuberculosis sensitivities of 84, 52 and 68% and specificities of 97, 97 and 93%, respectively. In addition, in children the TST shows higher test sensitivity when compared to the QFT-GIT. In immunologically compromized individuals the TST and QFT-GIT show only a weak sensitivity with a high sensitivity (Doan et al. (2018) PLOS ONE 12(11):e0188631).

In the field of infection recognition (discrimination of active disease and latent infection on the one hand versus patients without contact with mycobacterial pathogens on the other hand) a meta-analysis reports IGRAs to have sensitivities/specificities in a range of 73-83% and 49-79%, respectively (Sester et al. (2011) Eur. Resp. J. 37:100; World Health Organization, Tuberculosis IGRA TB Test Policy Statement, 2011).

Thus, there exists a need for a method, which allows a more reliable and automatable detection of mycobacterial infections.

In addition, within the last decade novel molecular immunodiagnostic tests have been developed based on RT-qPCR-based quantification of markers, which are produced by tuberculosis-specific memory T cells and/or antigen presenting cells in response to stimulation with tuberculosis antigens (WO2008028489A3, WO2012037937A2). Herein, relative quantification of CXCL10 mRNA by qPCR as claimed in WO2008028489A3 is almost equally efficient in detection of mycobacterial infection as the commercial (QFT-GIT) test (Blauenfeld et al. (2014) PLOS ONE 9:e105628). Divergent from the method described in the patent application WO2012037937A2 the present invention describes a RT-qPCR-based method for the discrimination of active tuberculosis and latent mycobacterial infection from non-infected individuals.

The problem to be solved by the present invention was thus to provide a more specific and/or sensitive method for detecting infection with pathogens causing tuberculosis. A further problem to be solved by the present invention was the provision of a method for detecting infection with pathogens causing tuberculosis which can be automatized. A further problem to be solved by the present invention was the provision of a method allowing a quick test result e.g. within about 4 to 6 hours. A further problem to be solved by the present invention was the provision of a method in which a blood sample can be directly used for detection.

The problem underlying the present invention is solved by the subject matter defined in the claims.

The following figures serve the purpose of illustrating the invention.

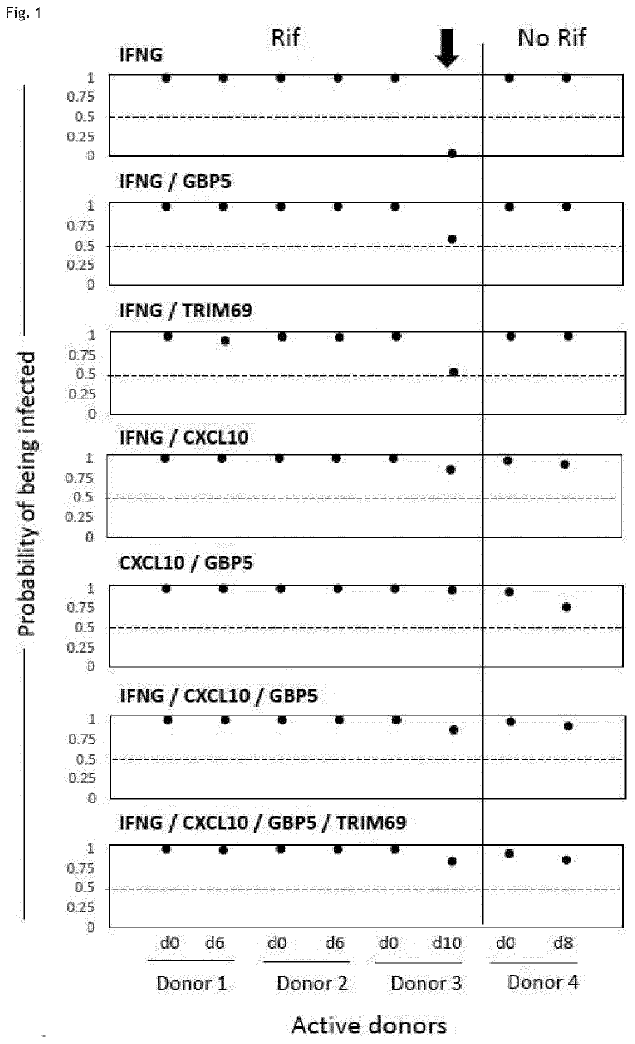

FIG. 1 shows a graph representing the probability of being infected of four active TB (ATB) donors treated (donors 1 to 3) or not treated (donor 4) with Rifampicin for the indicated days (d6 to d10) in comparison to a baseline time point (d0). Blood was drawn from patients with ATB at the two consecutive time points each. Whole blood samples were then stimulated with CFP10 and ESAT6, and RNA was isolated as described in example 1. The isolated RNA was used for cDNA synthesis and qPCR analysis as described in example 3. For all stimulated or unstimulated samples qPCRs on marker-genes IFNG, CXCL10, GBP5, and ncTRIM69, as well as on the housekeeping gene RPLP0 were performed. RPLP0 was used to normalize marker-gene expression and differences between stimulated and non-stimulated samples from one donor was used to calculate the fold change as described in example 4. Probability of being infected was determined using the blood-based classifier, as described in Example 6.

In the context of the present invention an “antigen” is preferably understood to be a protein, a polypeptide or a peptide, wherein said protein, polypeptide or peptide preferably encodes at least a part of or a complete pathogen causing tuberculosis. In addition, an antigen may be understood to be a RNA, DNA or an expression plasmid, wherein said nucleic acids encode at least a part, preferably a peptide, polypeptide or protein of least a part of or a complete pathogen causing tuberculosis. Preferably, the antigen is an antigen of a wild type pathogen causing tuberculosis but not of attenuated M. tuberculosis strains used for vaccination, in particular not of the BCG (Bacille Calmette-Guerin)-vaccine strain.

The term “sensitivity” as used herein refers preferably to the % of patients with active tuberculosis and latent mycobacterial infection (defined as “infected”) that are correctly classified as infected.

The term “specificity” as used herein refers preferably to the % of individuals with no previous contact with a pathogen causing tuberculosis as e.g. mycobateria (defined as “non-infected”) that are correctly classified as non-infected.

In the context of the present invention the term “polypeptide” is preferably understood to be a polymer of amino acids of any length. The phrase “polypeptide” comprises also the terms target epitope, epitope, peptide, oligopeptide, protein, polyprotein and aggregate of polypeptides. Furthermore, the expression “polypeptide” also encompasses polypeptides, which exhibit posttranslational modifications such as glycosylations, acetylations, phosphorylations, carbamoylations and similar modifications. In addition, the expression “polypeptide” is understood to refer also to polypeptides, which exhibit one or more analogues of amino acids, such as for example non-natural amino acids, polypeptides with substituted linkages as well as other modifications known in the prior art, irrespective thereof, whether they occur naturally or are of non-natural origin.

In the context of the present invention “reverse transcription quantitative real-time polymerase chain reaction, RT-qPCR” is preferably understood to be a method, which is based on the conventional polymerase chain reaction (PCR). In addition, RT-qPCR allows, besides amplification, in addition also a quantification of the target mRNA. For this purpose the total RNA is isolated from the material to be examined and incubated with an antigen and is isolated in comparison from unstimulated material or material incubated with an irrelevant antigen, and is then transcribed into cDNA in a subsequent reverse transcription reaction. By using specific primers the target sequence is then amplified in the qPCR. For quantification of the target sequence several methods may be applied.

The most simple way of quantification in RT-qPCR is using intercalating fluorescent dyes, such as SYBR green or EVA green. These dyes fit themselves in the double stranded DNA molecules, which arise during the elongation of the specific products. The detection always takes place at the end of the elongation by detecting the emitted light after excitation of the fluorescent dye. With increasing amount of PCR product more dye is incorporated, thus the fluorescent signal increases.

A further possibility of quantification in RT-qPCR is the use of sequence specific probes. There are hydrolysis (TaqMan) or hybridisation (Light-Cycler) probes. Hydrolysis probes are labelled at the 5′ end with a fluorescent dye and at the 3′ end with a so-called quencher. Due to the spatial proximity to the reporter dye the quencher is responsible for the quenching of the fluorescence signal and is cleaved off during the synthesis of the complementary DNA in the elongation phase. As soon as the fluorescent dye is excitated with a light source at the end of the elongation, light of a specific wave length is emitted, which may be detected.

Hybridisation probe systems consist of two probes, which bind to a target sequence next to each other. Both probes are labelled with a fluorescent dye. With a light source the first fluorescent dye at the 5′ end of the first probe is excited. The emitted light is then transferred via fluorescence resonance energy transfer (FRET) to the second fluorescent dye at the 3′ end of the second probe. Thereby the dye is excited, whereby light of a specific wave length is emitted, which may be detected. If in the course of the elongation of the complementary strand of the target sequence the first probe is degraded by the polymerase, the FRET may no more take place and the fluorescence signal subsequently decreases. In contrast to the afore-mentioned methods the quantification thus occurs here always at the beginning of the elongation process.

Frequently used fluorescent dyes are for example Fluophor 1, Fluorphor 2, aminocumarin, fluorescin, Cy3, Cy5, europium, terbium, bodipy, dansyl, naphtalene, ruthenium, tetramethylrhodamine, 6-carboxyfluorescein (6-FAM), VIC, YAK, rhodamine and Texas Red. Frequently used quenchers are for example TAMRA™, 6-carboxytetramethoylrhodamine, methyl red or dark quencher.

The term “real-time” refers preferably to a distinct measurement within each cycle of PCR, i.e. in “real-time”. The increase of the so-called target sequence correlates herein with the increase of the fluorescence from cycle to cycle. At the end of a run, which usually consists of several cycles, the quantification is then carried out in the exponential phase of the PCR on a basis of the obtained fluorescents signals. Hereby, the measurement of the amplification is usually done via Cq (quantification cycle) values, which described the cycle, in which the fluorescence rises for the first time significantly above the background fluorescence. The Cq value is determined on the one hand for the target nucleic acid and on the other hand for the reference nucleic acid. In this way it is possible to determine absolute or relative copy numbers of the target sequence.

In a preferred embodiment of the invention the normalisation of the gathered real-time PCR data (real-time PCR data) is performed by using a fixed reference value, which is not influenced by the conditions of the experiment, in order to achieve a precise gene expression quantification. For this purpose the expression of a reference gene is also measured in order to perform a relative comparison of amounts.

In the context of the present invention the expression reference gene may be understood as a sequence on mRNA level as well as on the level of genomic DNA. These may also be non-transcriptional active under the stimulation conditions according to the present invention or they correspond to non coding DNA regions of the genome. According to the invention a reference gene may also be a DNA or RNA added to the target gene sample. The highest criterion of a reference gene is that it is not altered in the course of the stimulation and by the conditions of the inventive method. The experimental results may thus be normalized with respect to the amount of template used in different samples. The reference gene allows thus the determination of the relative expression of a target gene. Examples for reference genes are 60S acidic ribosomal protein P0 (RPLP0), β-actin, glyceraldhyde-3-phosphate-dehydrogenase (GAPDH), porphobilinogen deaminase (PBGD) or tubulin.

In the context of the present invention the terms “RNA SEQ” or “RNA sequencing” preferably refers to a sequencing-based high-throughput approach for the qualitative and quantitative analysis of entire transcriptomes of organisms. Preferably, said approach is performed by sequencing fragmented cDNA, mapping the resulting sequences (“reads”) and comparing them to known genomes or transcriptomes. The reads may be assembled and annotated for example to protein databases or other transcriptomes. Quantification of the RNAs may be achieved by counting the corresponding fragments after annotation to a known genome or transcriptome or after de novo assembly and annotation to a protein-database. “RNA SEQ” preferably refers to “targeted RNA sequencing”, a method allowing the quantitative sequencing of selected RNA products, typically but not exclusively as described by Blomquist et al. (2013, PloS ONE 8(11): e79120; doi:10.1371/journal.pone.0079120). Martin et al. (2016, J. Vis. Exp. 114; doi: 10.3791/54090) or Gao et al. (2017, World of Gastroenterol. 23:2819).

In the context of the present invention “lymphatic tissue” is understood to be lymph nodes, spleen, tonsils as well as the lymphatic tissue of the gastrointestinal mucous membrane, such as peyers plaques, the lymphatic tissue of the respiratory organs and of the urinary tracts.

The term “% sequence identity” is generally understood in the art. Two sequences to be compared are aligned to give a maximum correlation between the sequences. This may include inserting “gaps” in either one or both sequences, to enhance the degree of alignment. A % identity may then be determined over the whole length of each of the sequences being compared (so-called global alignment), that is particularly suitable for sequences of the same or similar length, or over shorter, defined lengths (so-called local alignment), that is more suitable for sequences of unequal length. In the above context, an amino acid sequence having a “sequence identity” of at least, for example, 95% to a query amino acid sequence, is intended to mean that the sequence of the subject amino acid sequence is identical to the query sequence except that the subject amino acid sequence may include up to five amino acid alterations per each 100 amino acids of the query amino acid sequence. In other words, to obtain an amino acid sequence having a sequence of at least 95% identity to a query amino acid sequence, up to 5% (5 of 100) of the amino acid residues in the subject sequence may be inserted or substituted with another amino acid or deleted. Methods for comparing the identity and homology of two or more sequences are well known in the art and may for example be performed by a BLAST analysis. In addition, if reference is made herein to a sequence sharing “at least” at certain percentage of sequence identity, then 100% sequence identity are preferably not encompassed.

In a first object of the present invention it is envisaged to provide an in vitro method of detecting an infection with pathogens causing tuberculosis, the method comprises the steps of:
(a) contacting a first aliquot of a sample of an individual with at least one antigen of a pathogen causing tuberculosis,
b) incubating the first aliquot with the at least one antigen over a certain period of time,
c) detecting in the first aliquot and in a second aliquot of the sample of the individual at least two marker using reverse transcription quantitative real-time polymerase chain reaction (RT-qPCR) or RNA Sequencing (RNA-Seq), wherein the second aliquod has not been incubated with the at least one antigen, and wherein one of the at least two markers is IFN-γ or CXCL10 and the other of the at least two markers is either a distinct one of IFN-γ, or CXCL10 or one of ncTRIM69, GBP5, CTSS and IL19, and
d) comparing the detected marker(s) in the first aliquot with the detected marker(s) in the second aliquot.

The in vitro method of detecting an infection with pathogens causing tuberculosis according to the present invention is preferably an in vitro method for differentiating individuals being infected with pathogens causing tuberculosis and individuals being uninfected with pathogens causing tuberculosis. The method according to the present invention provides an improved detection of infection with tuberculosis pathogens, especially of individuals with active tuberculosis. The test allows the diagnosis of infection with tuberculosis pathogens and their differentiation from individuals without contact with tuberculosis pathogens. Individuals without contact with tuberculosis pathogens preferably include non vaccinated individuals without contact with tuberculosis pathogens and individuals being vaccinated against tuberculose, as e.g. BCG vaccinated individuals, which had no further contact with tuberculosis pathogens. Both people with latent infection and patients with active disease are detected. In a preferred embodiment also actively infected individuals under initiation of antibacterial therapy, e.g. with Rifampicin, are detected as having been in contact with a pathogen causing tuberculosis. The method according to the present invention does not allow distinguishing between individuals having a latent infection and individuals having active tuberculosis.

The method according to the present invention allows an improved detection of individuals with latent infection with pathogens causing tuberculosis and patients suffering from active tuberculosis and the discrimination from non vaccinated and preferably vaccinated, preferably BCG-vaccinated individuals, with no contact with a pathogen causing tuberculosis. This methodology has improved performance parameters compared to the commercially available tuberculin skin (PPT) and interferon gamma release (IGRA) tests and provides some operational advantages such as high analytical accuracy, rapid availability of test result and suitability for fully automated workflows. In addition, molecular immunodiagnostics require shorter incubation time compared to conventional protein based tests (4 to 6 hours instead of 16 to 24 hours).

Unexpected findings were the synergistic effects of the non coding regions of TRIM69 (ncTRIM69), GBP5, IL19 and to a lower extent CTSS with the IFN-g and/or CXCL10 marker applying RT-qPCR based read-out systems in individuals with latent infection and active tuberculosis, in particular prior to and during Rifampicin treatment. The method of the present invention allows detection of infection with tuberculosis pathogens with sensitivities and/or specificities ranging from app. 90 to up to 95%, more preferably up to 96%, 97%, 98% or 99% depending on the applied patient sample, marker combination and evaluation methodology.

According to the method of the present invention the at least two markers are selected as follows: One of the at least two markers is IFN-γ or CXCL10 and the other of the at least two markers is either a distinct one of IFN-γ or CXCL10 or one of ncTRIM69, CTSS, GBP5 and IL19. In other words this means that one of the at least two markers is IFN-γ or CXCL10 and the other of the at least two markers is either one of IFN-γ or CXCL10 with the provision that the at least two markers are not identical, or one of ncTRIM69, CTSS, GBP5 and IL19. An example for such a marker combination is a combination comprising or consisting of IFN-γ and CXCL10.

In a preferred embodiment of the present invention in step c) one of the at least two markers is IFN-γ or CXCL10 and the other of the at least two markers is one of ncTRIM69, GBP5, CTSS and IL19. Accordingly, in step c) preferably a marker combination is detected comprising or consisting of:
IFN-γ and GBP5
IFN-γ and ncTRIM69
IFN-γ and CTSS
IFN-γ and IL19
CXCL10 and GBP5
CXCL10 and ncTRIM69
CXCL10 and CTSS
CXCL10 and IL19

In a further embodiment, in step c) of the in vitro method as defined above, at least a third, optionally a fourth, optionally a fifth and optionally a sixth marker is detected, wherein the at least third, fourth, fifth or sixth marker is selected from the group consisting of: IFN-γ, CXCL10, GBP5, ncTRIM69, CTSS and IL19, with the provision that the first, second, third and optionally fourth, fifth and sixth marker are each distinct markers. Preferred examples for such marker combinations are combinations comprising or consisting of:
CXCL10, IL19, and ncTRIM69;
CTSS, IFN-γ, ncTRIM69
CTSS, IFN-γ, IL19, and ncTRIM69
CTSS, CXCL10, and ncTRIM69
IFN-γ, IL19, and ncTRIM69
CTSS, CXCL10, IL19, and ncTRIM69
IFN-γ, GBP5, CXCL10, and ncTRIM69
CXCL10, GBP5, IFN-γ, and CTSS
CTSS, CXCL10, GBP5, IFN-γ, and ncTRIM69
CXCL10, IFN-γ, IL19, and ncTRIM69
CTSS, CXCL10, IFN-γ, and ncTRIM69
CTSS, CXCL10, IFN-γ, IL19, and ncTRIM69
IFN-γ, GBP5, CXCL10, IL19, and ncTRIM69
CXCL10, IFN-γ, IL19, and GBP5
CTSS, CXCL10, IFN-γ, and IL19
CTSS, CXCL10, GBP5, IFN-γ, and IL19
CTSS, CXCL10, GBP5, IFN-γ, IL19, and ncTRIM69
CTSS, CXCL10, GBP5, and ncTRIM69
CXCL10, GBP5, IL19, and ncTRIM69
CTSS, GBP5, IFN-γ, and ncTRIM69
GBP5, IFN-γ, IL19, and ncTRIM69
CTSS, GBP5, IFN-γ, IL19, and ncTRIM69
CTSS, CXCL10, GBP5, IL19, and ncTRIM69
CTSS, IFN-γ, IL19, and ncTRIM69
CTSS, CXCL10, and ncTRIM69
IFN-γ, IL19, and ncTRIM69
CTSS, CXCL10, IL19, and ncTRIM69

In a further embodiment, in step c) of the in vitro method as defined above at least a third marker is detected wherein two of the at least three markers are IFN-γ, CXCL10 or GBP5 and the other of the at least three markers is either a distinct one of IFN-γ, CXCL10, or GBP5 or one of ncTRIM69, CTSS and IL19. Thus, in particular marker combinations are preferred which comprise or consist of one of the following combinations:
IFN-γ, GBP5, and CXCL10
IFN-γ, CXCL10, and CTSS
CXCL10, IFN-γ, and ncTRIM69
CXCL10, IFN-γ, and IL19
GBP5, IFN-γ, and ncTRIM69
CTSS, GBP5, and IFN-γ
IFN-γ, GBP5, and IL-19
CXCL10. GBP5, and ncTRIM69
CTSS, CXCL10, and GBP5
CXCL10, GBP5, and IL19

If the sample is or comprises blood, in particular whole blood or anticoagulated whole blood, the following marker combinations are particularly preferred:
IFN-γ, GBP5, CXCL10, IL19, and ncTRIM69
CXCL10. IFN-γ, IL19, and GBP5
CXCL10, GBP5, and ncTRIM69
CTSS, CXCL10, IFN-γ, and IL19
CTSS, CXCL10, GBP5, IFN-γ, and IL19
CTSS, CXCL10, GBP5, IFN-γ, IL19, and ncTRIM69
CTSS, CXCL10, GBP5, and ncTRIM69
CXCL10, IL19, and ncTRIM69
CXCL10, GBP5, IL19, and ncTRIM69
CTSS, CXCL10, and GBP5
IFN-γ, GBP5, and CXCL10
IFN-γ, GBP5, CXCL10, and ncTRIM69
CXCL10, GBP5, IFN-γ, and CTSS
IFN-γ, CXCL10, and CTSS
CTSS, CXCL10, GBP5, IFN-γ, and ncTRIM69
CXCL10, IFN-γ, and ncTRIM69
CXCL10, IFN-γ, and IL19
CXCL10, IFN-γ, IL19, and ncTRIM69
CTSS, CXCL10, IFN-γ, and ncTRIM69
CTSS, CXCL10, IFN-γ, IL19, and ncTRIM69
GBP5, IFN-γ, and ncTRIM69
CTSS, GBP5, and IFN-γ

If the sample comprises purified or isolated PBMC, the following marker combinations are particularly preferred:
CTSS, IFN-γ, and ncTRIM69
IFN-γ, GBP5, and CXCL10
IFN-γ, GBP5, CXCL10, and ncTRIM69
CXCL10, GBP5, IFN-γ, and CTSS
IFN-γ. CXCL10, and CTSS
CTSS, CXCL10, GBP5, IFN-γ, and ncTRIM69
CXCL10, IFN-γ, and ncTRIM69
CXCL10, IFN-γ, and IL19
CXCL10, IFN-γ, IL19, and ncTRIM69
CTSS, CXCL10, IFN-γ, and ncTRIM69
CTSS, CXCL10, IFN-γ, IL19, and ncTRIM69
GBP5, IFN-γ, and ncTRIM69
CTSS, GBP5, and IFN-γ

In another embodiment the present invention provides an in vitro method of detecting an infection with pathogens causing tuberculosis comprising the steps:
(a) contacting a first aliquot of a sample of an individual with at least one antigen of a pathogen causing tuberculosis,
b) incubating the first aliquot with the at least one antigen over a certain period of time,
c) detecting in the first aliquot and in a second aliquot of the sample of the individual at least one marker using quantitative PCR (qPCR), reverse transcription quantitative real-time polymerase chain reaction (RT-qPCR), RNA Sequencing (RNA-Seq), expression profiling and microarray, wherein the second aliquod has not been incubated with the at least one antigen, and wherein the at least one marker is ncTRIM69, and
d) comparing the detected marker(s) in the first aliquot with the detected marker(s) in the second aliquot.

In a preferred embodiment of the method according to the present invention, in which the at least one marker in step c) is ncTRIM69 (called TRIM-method in the following) at least a second marker is detected in step c) in the first aliquot and in the second aliquot, wherein the second marker is selected from the group consisting of: IFN-γ, CXCL10, GBP5, CTSS and IL19.

In a further preferred embodiment of the TRIM-method according to the present invention at least a second, a second and a third, a second, third and fourth marker, a second, third, fourth and fifth, or a second, third, fourth, fifth or sixth marker is detected in step c) in the first aliquot and in the second aliquot, wherein the second, and optionally third, fourth, fifth and sixth marker is selected from the group consisting of: IFN-γ, CXCL10, GBP5, CTSS and IL19 with the provision that the second, and optionally third, fourth, fifth and sixth marker are each distinct markers.

In a further preferred embodiment of the TRIM-method according to the present invention a marker combination is detected in step (c), wherein the marker combination comprises or consists of one of the following combinations:
IL19 and ncTRIM69
IFN-γ and ncTRIM69
IFN-γ, IL19 and ncTRIM69
IFN-γ, IL19 and ncTRIM69
GBP5 and ncTRIM69
GBP5, IL19 and ncTRIM69
GBP5, IFN-γ and ncTRIM69
GBP5, IFN-γ, IL19 and ncTRIM69
CXCL10 and ncTRIM69
CXCL10, IL19 and ncTRIM69
CXCL10, IFN-γ and ncTRIM69
CXCL10, IFN-γ, IL19 and ncTRIM69
CXCL10, GBP5 and ncTRIM69
CXCL10, GBP5, IL19 and ncTRIM69
CXCL10, GBP5, IFN-γ and ncTRIM69
CXCL10, GBP5, IFN-γ, IL19 and ncTRIM69
CTSS and ncTRIM69
CTSS, IL19 and ncTRIM69
CTSS, IFN-γ and ncTRIM69
CTSS, IFN-γ, IL19 and ncTRIM69
CTSS, GBP5 and ncTRIM69
CTSS, GBP5, IL19 and ncTRIM69
CTSS, GBP5, IFN-γ and ncTRIM69
CTSS, GBP5, IFN-γ, IL19 and ncTRIM69
CTSS, CXCL10 and ncTRIM69
CTSS, CXCL10, IL19 and ncTRIM69
CTSS, CXCL10, IFN-γ and ncTRIM69
CTSS, CXCL10, IFN-γ, IL19 and ncTRIM69
CTSS, CXCL10, GBP5 and ncTRIM69
CTSS, CXCL10, GBP5, IL19 and ncTRIM69
CTSS, CXCL10, GBP5, IFN-γ and ncTRIM69
CTSS, CXCL10, GBP5, IFN-γ, IL19 and ncTRIM69

The following embodiments are preferred embodiments of all methods according to the present invention including the first described method according to the present invention and the TRIM method. In a preferred embodiment the detection of an infection with pathogens causing tuberculosis is a differentiation of individuals having been in contact with a pathogen causing tuberculosis and individuals having not been in contact with a pathogen causing tuberculosis. Individuals having been in contact with pathogens causing tuberculosis comprise preferably individuals having acute tuberculosis, active tuberculosis, which preferably requires treatment, latent infection with pathogens causing tuberculosis and individuals in which tuberculosis have been successfully treated i.e. the pathogens causing tuberculosis have been successfully killed or combated by therapy. In a preferred embodiment also actively infected individuals under initiation of antibacterial therapy e.g. with Rifampicin are detected as having been in contact with a pathogen causing tuberculosis. Preferably, individuals having not been in contact with pathogens causing tuberculosis comprise individuals having been vaccinated against tuberculosis, in particular individuals having been vaccinated with the Bacillus Calmette-Guérin (BCG) vaccination strain. Such individuals may also called BCG-vaccinated individuals. The individual may be a human or an animal.

According to the invention it is contemplated that the method of detecting an infection with pathogens causing tuberculosis comprises the step of providing a sample of an individual. Said sample is preferably a liquid sample as e.g. a whole blood sample. In the context of the present invention “providing” is understood to imply that an aliquot of the sample is already present in a container. “Providing” may also mean according to the invention, that the aliquot of the sample is directly provided from a patient, for instance by sampling blood. The inventive method envisages that the first aliquot is stimulated with at least one antigen, while the second aliquot remains unstimulated. However, said second aliquot may be incubated or even stimulated by a mock control. A mock treatment is a sham treatment of reaction or incubation approaches which serves as a control experiment. In a mock treatment the mock control is preferably treated in the same way as the parallel approach but without one or more active components. Said mock control may comprise antigens but no antigens of pathogens causing tuberculosis and/or no antigens causing the specific reaction which is caused by pathogens causing tuberculosis. All in all it is thus envisaged, that the first and second aliquot are identical except for the contact with the antigen/s, i.e. the antigens of pathogens causing tuberculosis which are used in step (a) of the methods according to the present invention. However, instead of the antigen(s) of pathogens causing tuberculosis one ore more different antigens, which are not of pathogens causing tuberculosis and/or do not cause the specific reaction which is caused by pathogens causing tuberculosis may be added to the second aliquot e.g. for stimulating the components of the second aliquot. Preferably, the first and second aliquod are identical except for the added stimulants and antigens, respectively. Hence, the second unstimulated aliquot serves as a kind of calibrator. The quantification is thus performed relative to the calibrator. For the determination and quantification of the marker it is envisaged, that the amount of marker in the first stimulated aliquot is divided by the amount of the marker in the second unstimulated aliquot. Thus, an n-fold difference in amount of the marker of the first stimulated aliquot relative to the calibrator, i.e. the second unstimulated aliquot, is detected. The inventive method represents a method which is exclusively carried out ex vivo.

In a preferred embodiment the sample is or comprises a body fluid. The body fluid may be blood, lymph, a bronchial lavage, or a suspension of lymphatic tissue. The blood is preferably whole blood or anticoagulated whole blood. Also preferred are embodiments in which the sample comprises isolated cells of the above listed body fluids. Particularly preferred is a sample of an isolated PBMC or a purified PBMC population, preferably a PBMC population isolated from whole blood, or cells isolated from a bronchial lavage. Cells isolated from a bronchial lavage may for example be obtained by applying density gradient centrifugation using Ficoll-Paque media. Isolated cells may be resuspended and optionally cultured in a suitable medium as e.g. serum-free medium or serum containing medium.

The sample of an individual can be a previously obtained from a human or an animal patient. Preferably, the method according to the present invention is performed about 0 to about 48 hours, more preferably about 0 to about 36 hours, or about 1 to about 10 hours or about 3 to about 8 hours, or about 0.5 hours to about 8 hours or about 0.5 hours to about 24 hours after the sample of the individual was obtained. Most preferably, the method according to the present invention is performed at a time period of less than or equal to 8 hours after the sample of the individual was obtained, i.e. about 0 to 8 hours after the sample of the individual was obtained. After the sample was obtained from the individual, the sample is preferably stored at a temperature above 0° C., more preferably at a temperature of about 0° C. to about 50° C., about 4° C. to about 40° C., about 10° C. to about 35° C. or about 16° C. to about 30° C., or about 18° C. to about 25° C., or at about room temperature.

In a preferred embodiment the at least one antigen of a pathogen causing tuberculosis is a peptide, oligopeptide, a polypeptide, a protein, a RNA or a DNA. According to the invention the antigen may furthermore preferably be a fragment, a cleavage product or a piece of an oligopeptide, of a polypeptide, of a protein, of an RNA or of a DNA. In a further preferred embodiment, the at least one antigen of a pathothen causing tuberculosis is a protein, in particular having a length of about 4 kDa to about 100 kDa, or about 5 kDa to about 90 kDa.

In a preferred embodiment of the method according to the present invention step (a) comprises contacting a first aliquot of a sample of an individual with two, three, four, five, six, seven, eight, nine or ten antigens of a pathogen causing tuberculosis. The aliquot in step (a) may also be contacted with 11, 12, 13, 14, 15, 16, 17, 18, 19, 20, 21, 22, 23, 24, 25, 26, 27, 28, 29 or 30 or with a pool of antigens comprising about 10 to about 100, about 20 to about 100, about 30 to about 100, about 40 to about 100 or about 50 to about 100 antigens. If more than one antigen is used, all antigens are preferably distinct antigens. The distinct antigens may be derived from one or more different pathogens causing tuberculosis. They may also derive from the same pathogen causing tuberculosis. If 3 or more than 3 distinct antigens are used some of the antigens may derive from the same pathogen and the other may derive from different pathogens causing tuberculosis. A pool of antigens comprises preferably peptides as antigens.

In a preferred embodiment the at least one antigen and optionally the further antigens as described above are selected from the group consisting RD-1 antigens, ESAT-6, CFP10, TB7.7, Ag 85, HSP-65, Ag85A, Ag85B, MPT51, MPT64, TB10.4, Mtb8.4, hspX, Mtb12, Mtb9.9, Mtb32A, PstS-1, PstS-2, PstS-3, MPT63, Mtb39, Mtb41, MPT83, 71-kDa, PPE68 and LppX. Especially preferred antigens are ESAT-6, CFP-10, TB 7.7, Ag 85, HSP 65 and other RD-1 antigens. RD1-1 antigens are preferably the following antigens: Rv3871, Rv3872, Rv3873, CFP-10, ESAT-6, Rv3876, Rv3878, Rv3879c and ORF-14.

Alternatively or in addition, the antigens may be also selected from the group consisting H1-hybrid, AlaDH, Ag85B, Pst1S, Ag85, ORF-14, Rv0134, Rv0222, Rv0934, Rv1256c, Rv1514c, Rv1507c, Rv1508c, Rv1511, Rv1512, Rv1516c Rv1766 Rv1769 Rv1771, Rv1860, Rv1974 Rv1976c Rv1977, Rv1980c, Rv1982c, Rv1984c, Rv1985c, Rv2031c, Rv2074, Rv2780, Rv2873 Rv3019c, Rv3120, Rv3615c Rv3763, Rv3871, Rv3872, Rv3873, Rv3876, Rv3878, Rv3879c, Rv3804c, Rv3873, Rv3878, Rv3879c or a polypeptide mixture, such as tuberculin PPD.

Alternatively or in addition, the antigens may be selected from the group consisting of Rv3879c, Rv1508c, Rv3876, Rv1979c, Rv2655c, Rv1582c, Rv1586c, Rv3877, Rv2650c, R1576c, Rv1256c, Rv3618, Rv2659, cRv1770, Rv1771, Rv1769, Rv3428c, Rv1515c, Rv1511, Rv1512, Rv1977, Rv1985c, Rv0134, Rv1509, Rv3427c, Rv2646, Rv1041, cRv1507c, Rv1980c, Rv1514c, Rv1190, Rv3878, Rv1969, Rv1975, Rv1968, Rv1971, Rv3873, Rv2652c, Rv2651c, Rv1585c, Rv1577c, Rv1972, Rv1507A, Rv1506c, Rv1966, Rv1973, Rv1573. Rv1578c, Rv1974, Rv1575, Rv2645, Rv1987, Rv1970, Rv2074, Rv1976c, Rv2073c, Rv2810c, Rv1581c, Rv3136A, Rv2548A, Rv3098A, Rv2231A, Rv2647, Rv1772, Rv1508A, Rv2658c, Rv1767, Rv2063A, Rv1954, ARv1583c, Rv2656c, Rv0724A, Rv3875, Rv2348c, Rv0222, Rv2653c, Rv1580c, Rv1579c, Rv1766, Rv1366A, Rv3874, Rv0061c, Rv1768, Rv0397A, Rv1991A, Rv2274A, Rv3617, Rv1574, Rv3350c, Rv1984c, Rv2801A, Rv3872, Rv2657c, Rv1983, Rv2142A, Rv1967, Rv2862A, Rv3190A, Rv2237A, Rv2468A, Rv1982A, Rv1982c, Rv1584c, Rv0691A, Rv2395A, Rv2654c, Rv2231B, Rv1257c, Rv2395B, Rv1516c, Rv0186A, Rv0530A, Rv0456B, Rv3120, Rv3738c, Rv3121, Rv3426, Rv3621c, Rv0157A, Rv2349c, Rv1965, Rv3508, Rv3514, Rv0500B, Rv1978, Rv2350c, Rv2351c, Rv1986, Rv3599c, Rv2352c, Rv1255c, Rv2356c, Rv2944, and Rv3507.

Particularly preferred is an embodiment of the present invention, wherein step (a) comprises contacting a first aliquot of a sample of an individual with two antigens, in particular with CFP10 and ESAT6. Also particularly preferred is an embodiment of the present invention, wherein step (a) comprises contacting a first aliquot of a sample of an individual with three antigens, in particular with CFP10, ESAT6 and TB7.7.

In a preferred embodiment of the present invention the period of time for contacting in step a) and incubation in step b) is about 0.5 to about 36 hours, more preferably about 1 hours to about 24 hours or about 3 hours to about 24 hours, more preferably about 30 min to about 8 hours, or about 2 hours to about 8 hours, or about 2 hours to about 7 hours, or about 3 hours to about 6 hours, or over night, preferably about 8 hours to about 36 hours, or about 10 hours to about 30 hours or about 12 to about 28 hours or about 14 to about 26 hours or about 16 to about 24 hours or about 30 minutes, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or about 36 hours. The period of time for contacting in step a) and incubation in step b) is the time during which the sample of the individual is contacted and thus stimulated with the at least one antigen. Said stimulation is most preferably performed over night or in a time period of about 14 hours to about 24 hours, more preferably of about 15 hours to about 23 hours. Preferably, the time period for the stimulation over night or in a time period of about 14 hours to about 24 hours, more preferably of about 15 hours to about 23 hours is combined with a time period of less than or equal to 8 hours, or about 0 hours to about 8 hours after the sample of the individual was obtained.

Preferably, the pathogen causing tuberculosis is Mycobacterium tuberculosis, Mycobacterium bovis (ssp. bovis and caprae), Mycobacterium africanum, Mycobacterium microti, Mycobacterium canetti and Mycobacterium pinnipedii.

In a preferred embodiment of the invention RT-qPCT is used for detecting the marker/s in step c). If RT-qPCT is used the gathered real-time PCR data (real-time PCR data) are preferably normalized by using a fixed reference value, which is not influenced by the conditions of the experiment, in order to achieve a precise gene expression quantification. For this purpose the expression of a reference gene is also measured in order to perform a relative comparison of amounts. The reference gene is preferably measured in the first and in the second aliquod. Preferred reference genes are 60S acidic ribosomal protein P0 (RPLP0), β-actin, glyceraldhyde-3-phosphate-dehydrogenase (GAPDH), porphobilinogen deaminase (PBGD) and tubulin.

In a further preferred embodiment step d) is performed by analysing a detectable change in marker expression in the first aliquod in comparison to the second aliquod, preferably above a certain threshold. Alternatively, step d) may be performed by a classifier analysis or classification method, by fold change analysis, or by analyzing a change of the absolute amount of marker mRNA in the first and the second aliquod. Preferably, step d) of the method according to the present invention comprises (i) the comparison of the amount of the detected marker(s) of the first aliquot with the amount of the detected marker(s) of the second aliquot, (ii) a fold change analysis of the detected marker(s) in the first and in the second aliquot, or a combination of (i) and (ii). The comparison of the detected marker(s) in the first aliquot with the detected marker(s) in the second aliquot is preferably not performed by subtracting the detected marker(s) level in the second aliquot from the detected marker(s) level in the first aliquot. In fact, the comparison of the detected marker(s) is preferably performed by dividing the amount of marker in the first aliquot (the stimulated aliquot) by the amount of marker in the second aliquot (the unstimulated aliquot). Thus, an n-fold difference in amount of the marker of the first aliquot relative to the second aliquot is detected. Such an analysis is called fold change analysis.

In a preferred embodiment a difference in marker expression in the first and second aliquot is indicative that the individual is infected with pathogens causing tuberculosis or has been in contact with a pathogen causing tuberculosis. The difference in marker expression may be a detectable change in marker expression in the first aliquod in comparison to the second aliquod, preferably above a certain threshold and/or may be determined by a classifier analysis, by fold change analysis and/or by a change of the absolute amount of marker mRNA in the first and in the second aliquod. Particularly preferred is a combination of fold change analysis and random forest analysis.

In a preferred embodiment the method according to the present invention comprises an additional step (e) of detecting an infection with pathogens causing tuberculosis and/or differentiating individuals being infected with pathogens causing tuberculosis and individuals being uninfected with pathogens causing tuberculosis based on the comparison performed in step (d). Said additional step (e) may comprise the step of determining whether the individual is infected with pathogens causing tuberculosis or has been in contact with pathogens causing tuberculosis. In particular, step (e) may comprise the indication whether it is likely that the individual of which the sample was obtained is infected with pathogens causing tuberculosis or has been in contact with a pathogen causing tuberculosis. Preferably, step (e) may comprise calculating the probability that the person from which the sample was obtained is infected with pathogens causing tuberculosis or has been in contact with pathogen causing tuberculosis. Alternatively or in addition, step (e) may comprise the calculation of the probability that the person from which the the sample was obtained is not infected with pathogens causing tuberculosis or has not been in contact with pathogen causing tuberculosis. Step (e) can be performed subsequent to step (d) or may be incorporated into step (d).

Step d) and optionally (e) may be performed by a classification method as e.g. artificial neural networks, logistic regression, decision trees, Random Forest, Least Absolute Shrinkage and Selection Operator (LASSO), support vector machines (SVMs), threshold analysis, linear discriminant analysis, k-Nearest Neighbor (kNN), Naive Bayes, Bayesian Network, or any other method developing classification models known in the art.

In a preferred embodiment a Random Forest approach is performed as the classification method. Random Forests (Breiman 2001. “Random Forests”. Machine Learning. 45: 5-32; doi:10.1023/A:1010933404324) are an ensemble learning method for classification, regression and other tasks, that operate by constructing a multitude of decision trees at training time and outputting the class that is the mode of the classes (classification) or mean prediction (regression) of the individual trees. Random Forests correct for decision trees' habit of overfitting to their training set.

The random Forest approach can be performed by a basic Random Forest approach or by a probability Forest approach. The basic Random Forest approach denotes the original Random Forest implementation by Leo Breiman (2001, Machine Learning. 45 (1): 5-32; doi:10.1023/A:1010933404324) and the package ranger software may be used to perform this kind of Random Forest training and application. The probability Forest approach is based on the implementation of Random Forest proposed by Malley et al. (2012, Methods Inf Med 51:74-81; http://dx.doi.org/10.3414/ME00-01-0052) for probability estimation. The package ranger may be used to perform probability Forest training and application.

In order to get smoother probability estimations, the probability Forests were parametrized as follows: number of trees=1e3, minimal node size=5, split rule=“extratrees” with number of random split set to 5, and number of variables to possibly split at in each node set to 1. Generating classifiers with smoother probability estimations has also the aim to generate classifiers boundaries that will be more similar to those that would have been generated by a human process and limit overfitting. This corresponds to the following parameter setting in package ranger: number of trees (num.trees)=1e3, minimal node size (min.node.size)=5, split rule=“extratrees”, with the number of random splits (num.random.splits) set to 5 and the number of variables to possibly split at (mtry) set to 1. The use of Extra Trees (Geurts et al., 2006, Machine Learning. 63: 3-42; doi:10.1007/s10994-006-6226-1) is essentially motivated by the fact that resulting models are thus smoother than the piecewise constant ones obtained with other random forest implementations.

Practically, Random Forest classifiers may be established by using the software R [3.5.0] in combination with the packages ranger [0.9.0], readxl [1.1.0], stringr [1.3.0] and mlr [2.12.1]. The measurements of samples (as fold-change of antigen stimulation) were log 2-transformed before training using the function ranger( ), with the parameters described above.

In a particularly preferred embodiment of the present invention a combination of fold change analysis and random forest analysis is performed.

If the difference in marker expression in the first and second aliquot is indicative that the individual is infected with pathogens causing tuberculosis, the method according to the present invention may further comprise a step of administering a treatment to said individual. Preferably, said treatment comprises administering to the individual an amount of a therapeutic agent or a combination of therapeutic agents effective to treat tuberculosis. As needed, said therapeutic agent or combination of therapeutic agents is preferably effective to treat active tuberculosis or latent infection with pathogens causing tuberculosis or both.

Thus, in a further embodiment the present invention refers to a method of detecting an infection with pathogens causing tuberculosis and/or a method of treating and/or preventing tuberculosis, said method comprises:
(a) contacting a first aliquot of a sample of an individual with at least one antigen of a pathogen causing tuberculosis, and
b) incubating the first aliquot with the at least one antigen over a certain period of time, and
c1) detecting in the first aliquot and in a second aliquot of the sample of the individual at least two marker using reverse transcription quantitative real-time polymerase chain reaction (RT-qPCR) or RNA Sequencing (RNA-Seq), wherein the second aliquod has not been incubated with the at least one antigen, and wherein one of the at least two markers is IFN-γ or CXCL10 and the other of the at least two markers is either a distinct one of IFN-γ, or CXCL10 or one of ncTRIM69, GBP5, CTSS and IL19, or
c2) detecting in the first aliquot and in a second aliquot of the sample of the individual at least one marker using quantitative PCR (qPCR), reverse transcription quantitative real-time polymerase chain reaction (RT-qPCR). RNA Sequencing (RNA-Seq), expression profiling and microarray, wherein the second aliquod has not been incubated with the at least one antigen, and wherein the at least one marker is ncTRIM69, and
d) comparing the detected marker(s) in the first aliquot with the detected marker(s) in the second aliquot, and
e) evaluating whether the difference in marker expression in the first and second aliquot is indicative that the individual is infected with pathogens causing tuberculosis,
f) administering an effective amount of a therapeutic agent or a combination of therapeutic agents effective to treat tuberculosis to the individual evaluated to be infected with pathogens causing tuberculosis.

In a further preferred embodiment all preferred combinations of markers described above can be used in step c1) and c2), respectively.

The evaluation whether the difference in marker expression in the first and second aliquot is indicative that the individual is infected with pathogens causing tuberculosis may be performed by detecting an infection with pathogens causing tuberculosis in accordance with the present invention as described above.

In a further embodiment the present invention refers to a method of treating and/or preventing tuberculosis, said method comprises: administering an effective amount of a therapeutic agent or a combination of therapeutic agents effective to treat tuberculosis to an individual diagnosed to be infected with pathogens causing tuberculosis, wherein the respectively diagnosed individual has been diagnosed by the method according to the present invention as described herein. Before said individual is treated in accordance with the present invention said individual may be diagnosed in a second subsequent diagnosis step (i) to have a latent infection with pathogens causing tuberculosis, (ii) to suffer from an active tuberculosis infection or (iii) to have been in contact with pathogens causing tuberculosis, wherein the pathogens have successfully been killed or combated. Said second subsequent diagnosis step may be performed as known in the art and described herein.

Therapeutic agent(s) effective to treat and/or prevent tuberculosis may comprise therapeutic agents which are effective to kill, eliminate and/or neutralize pathogens causing tuberculosis and/or therapeutic agents which are effective in supporting the immune system of the individual to kill, eliminate and/or neutralize pathogens causing tuberculosis. Examples for suitable therapeutic agents are Rifapentine (RPT), Rifampin (RIF), Isoniazid (INH), Ethambutol (EMB) and Pyrazinamide (PZA), Rifabutin, Pyrazinamide, Ethambutol, Cycloserine, Ethionamide, Streptomycin, Amikacin/kanamycin, Capreomycin, Para-amino salicylic acid, Levofloxacin and Moxifloxacin. Said therapeutic agents may be administered alone or in combination with each other or in combination with further suitable therapeutic agents. In particular, a combination of Isoniazid and Rifapentine or a combination of Isoniazid, Rifampin, Pyrazinamide and Ethambutol is preferred.

If the difference in marker expression in the first and second aliquot is indicative that the individual is infected with pathogens causing tuberculosis, the method according to the present invention may comprise prior to the treating step a step of performing a differential diagnosis. Said differential diagnosis comprises preferably the step of determining whether the infected individual suffers from a latent infection with pathogens causing tuberculosis, an active tuberculosis, or has been in contact with pathogens causing tuberculosis, wherein the pathogens have successfully been killed or combated. Said differential diagnosis may for example be performed as described in the following publications: Lewinsohn et al. “Official American Thoracic Society/Infectious Diseases Society of America/Centers for Disease Control and Prevention Clinical Practice Guidelines: Diagnosis of Tuberculosis in Adults and Children”, CID 2016; 00(0):1-33; “Bericht zur Epidemiologic der Tuberkulose in Deutschland für 2016” provided by Robert Koch Institut; and Seybold, Ulrich, “Latente Tuberkulose—Infektion and Immunschwäche”, HIV&more 2/2016.

Individuals with a latent infection with pathogens causing tuberculosis usually do not have symptoms and they cannot spread tuberculosis bacteria to other. However, there is a risk that latent tuberculosis bacteria become active in the body and multiply. Thus, individuals having such a latent infection may for example be treated by the following Latent TB Infection Treatment Regimens published by the Centers for Disease Control and Prevention (CDC):

| Drugs | Duration | Interval |
|---|---|---|
| Isoniazid and Rifapentine | 3 months | Once weekly |
| Rifampin | 4 months | Daily |
| Isoniazid | 6 months | Daily or twice weekly |
| Isoniazid | 9 months | Daily or twice weekly |

When TB bacteria become active (multiplying in the body) and the immune system is not able to stop the bacteria from growing, this is called TB (tuberculosis) disease or active tuberculosis. Individuals having active tuberculosis may for example be treated by the following TB Infection Treatment Regimens published by the Centers for Disease Control and Prevention (CDC):

| | INTENSIVE PHASE | | CONTINUATION PHASE | | |
|---|---|---|---|---|---|
| | | Interval and Dose | | Interval and Dose | Range of Total |
| Regimen | Drugs | (minimum duration) | Drugs | (minimum duration) | Doses [mg] |
| 1 | INH | 7 days/week for 56 | INH | 7 days/week for 126 | 182 to 130 |
| | RIF | doses (8 weeks) | RIF | doses (18 weeks) | |
| | PZA | or | | or | |
| | EMB | 5 days/week for 40 | | 5 days/week for 90 | |
| | | doses (8 weeks) | | doses (18 weeks) | |
| 2 | INH | 7 days/week for 56 | INH | 3 times weekly for 54 | 110 to 94 |
| | RIF | doses (8 weeks) | RIF | doses (18 weeks) | |
| | PZA | or | | | |
| | EMB | 5 days/week for 40 | | | |
| | | doses (8 weeks) | | | |
| 3 | INH | 3 times weekly for 24 | INH | 3 times weekly for 54 | 78 |
| | RIF | doses (8 weeks) | RIF | doses (18 weeks) | |
| | PZA | | | | |
| | EMB | | | | |
| 4 | INH | 7 days/week for 14 doses then | INH | Twice weekly for 36 | 62 |
| | RIF | twice weekly for 12 doses | RIF | doses (18 weeks) | |
| | PZA | | | | |
| | EMB | | | | |

Alternatively, individuals may be treated by tuberculosis treatment methods known in the art as e.g. described in Nahid et al. (“Official American Thoracic Society/Centers for Disease Control and Prevention/Infectious Diseases Society of America Clinical Practice Guidelines: Treatment of Drug-Susceptible Tuberculosis”, ATS/TS/CDC/IDSA Clinical Practice Guidelines for Drug-Susceptible TB⋅CID 2016:63 (1 October), e147-e195).

The marker IFN-γ is well known in the art and is e.g. secreted by specifically restimulated antigen-specific memory T cells, in particular Th-1 cells and cytotoxic T cells. Multiple variants of IFN-γ are known in the art. Preferably, the marker IFN-γ is human IFN-γ. In one embodiment of the present invention the marker IFN-γ is encoded by a nucleic acid molecule comprising a nucleic acid sequence according to SEQ ID NO:1 or a functional variant thereof. Preferably, a IFN-γ functional variant may comprise a nucleic acid sequence having at least 60%, more preferably 70%, 80% or 90% sequence identity with the sequence of SEQ ID NO: 1. Preferably, a functional variant is a variant which expression is altered if the method according to the present invention is performed with a sample obtained from an individual having acute tuberculosis. The alteration of expression is preferably above a certain threshold, more preferably above 1.1, as described in the Examples. The term “IFN-γ” may be used interchangeable with the terms “INF-g”, “INFG”, “INF-gamma” and “INF-”, IFN-g”, “IFNG”, “IFN-gamma” and “IFN-.

In RT-qPCR any suitable primer that specifically binds to nucleic acids of IFN-γ may be used for detecting IFN-γ. Examples for suitable primers are nucleotides comprising a nucleic acid sequence according to SEQ ID NO: 2 and 3. Preferably, in addition to the primers a probe that specifically binds to nucleic acids of IFN-γ is used. For example a nucleic acid sequence comprising a sequence according to SEQ ID NO: 4 may be used as a probe. Said probe may comprise a fluorescence dye such as Bodipy TMR (BoTMR) (Invitrogen) and/or quencher.

The marker CXCL-10 is also known as IP-10 and is a small chemokine expressed by APCs and a main driver of proinflammatory immune responses. CXCL-10 is expressed by cells infected with viruses and bacteria, but can also be induced at high levels as part of the adaptive immune response. In this case, CXCL-10 secretion is initiated when T cells recognize their specific peptide presented on the APC. IP-10 secretion appears to be driven by multiple signals, mainly T-cell-derived IFN-g, but also IL-2, IFN-α, IFN-b, IL-27, IL-17, IL-23, and autocrine APC-derived TNF and IL-1b. Multiple variants of CXCL-10 are known in the art. Preferably, the marker CXCL-10 is human CXCL-10. In one embodiment of the present invention the marker CXCL-10 is encoded by a nucleic acid molecule comprising a nucleic acid sequence according to SEQ ID NO: 5 or a functional variant thereof. Preferably, a CXCL-10 functional variant may comprise a nucleic acid sequence having at least 60%, more preferably 70%, 80% or 90% sequence identity with the sequence of SEQ ID NO: 5. Preferably, a functional variant is a variant which expression is altered if the method according to the present invention is performed with a sample obtained from an individual having acute tuberculosis. The alteration of expression is preferably above a certain threshold, more preferably above 1.1, as described in the Examples.

In RT-qPCR any suitable primer that specifically binds to nucleic acids of CXCL-10 may be used for detecting CXCL-10. Preferably, in addition to the primers a probe that specifically binds nucleic acids of CXCL-10 or a functional fragment thereof is used. For example the commercial Primer probe ThermoFisher (exon 1/2 boundary)=Hs00171042_m1 may be used.

The marker GBP5 belongs to the family of IFN-γ-induced p65 GTPases, which are well known for their high induction by proinflammatory. The family of guanylate-binding proteins was originally identified by its ability to bind to immobilized guanine nucleotides with similar affinities for GTP, GDP and GMP. GBP5 protein highly expressed in mononuclear cells Loss of GBP5 function in a knockout mouse model results in impaired host defense and inflammatory response as GBP5 facilitates nucleotide-binding domain and leucine-rich repeat containing gene family, pyrin domain containing 3 (NLRP3)-mediated a member of the IFN-inducible subfamily of guanosine triphosphatases (GTPases) that play key roles in cell-intrinsic immunity against diverse pathogens. GBP5 promoted selective NLRP3 inflammasome responses to pathogenic bacteria and soluble but not crystalline inflammasome priming agents. Multiple variants of GBP5 are known in the art. Preferably, the marker GBP5 is human GBP5. In one embodiment of the present invention the marker GBP5 is encoded by a nucleic acid molecule comprising a nucleic acid sequence according to SEQ ID NO: 6 or a functional variant thereof. Preferably, a GBP5 functional variant may comprise a nucleic acid sequence having at least 60%, more preferably 70%, 80% or 90% sequence identity with the sequence of SEQ ID NO: 6. Preferably, a functional variant is a variant which expression is altered if the method according to the present invention is performed with a sample obtained from an individual having acute tuberculosis. The alteration of expression is preferably above a certain threshold, more preferably above 1.1, as described in the Examples.

In RT-qPCR any suitable primer that specifically binds to nucleic acids of GBP5 may be used for detecting GBP5. Preferably, in addition to the primers a probe that specifically binds to nucleic acids GBP5 is used. For example the commercial Primer probe ThermoFisher (exon 8/9 boundary)=Hs00369472_m1 may be used.

The marker IL-19 is a cytokine that belongs to the IL-10 cytokine subfamily. This cytokine is found to be preferentially expressed in monocytes. Its expression is up-regulated in monocytes following stimulation with granulocyte-macrophage colony-stimulating factor (GM-CSF), lipopolysaccharide, or Pam3CSK4. Multiple variants of IL-19 are known in the art. Preferably, the marker IL-19 is human IL-19. In one embodiment of the present invention the marker IL-19 is encoded by a nucleic acid molecule comprising a nucleic acid sequence according to SEQ ID NO: 7 or a functional variant thereof. Preferably, a IL-19 functional variant may comprise a nucleic acid sequence having at least 60%, more preferably 70%, 80% or 90% sequence identity with the sequence of SEQ ID NO:7. Preferably, a functional variant is a variant which expression is altered if the method according to the present invention is performed with a sample obtained from an individual having acute tuberculosis. The alteration of expression is preferably above a certain threshold, more preferably above 1.1, as described in the Examples.

In RT-qPCR any suitable primer that specifically binds to nucleic acid molecules of IL-19 may be used for detecting IL-19. Preferably, in addition to the primers a probe that specifically binds to nucleic acid molecules of IL-19 is used. For example the commercial Primer probe ThermoFisher (exon 4/5 boundary)=Hs00604657_m1 may be used.

The marker CTSS—a shortcut of Cathepsin S—is a lysosomal enzyme that belongs to the papain family of cysteine proteases. While a role in antigen presentation has long been recognized, it is now understood that cathepsin S has a role in itch and pain, or nociception. Cathepsin S is expressed by antigen presenting cells including macrophages, B-lymphocytes, dendritic cells, microglia and by some epithelial cells. Its expression is markedly increased in human keratinocytes following stimulation with interferon-gamma and its expression is elevated in psoriatic keratinocytes due to stimulation by proinflammatory factors. Multiple variants of CTSS are known in the art. Preferably, the marker CTSS is human CTSS. In one embodiment of the present invention the marker CTSS is encoded by a nucleic acid molecule comprising a nucleic acid sequence according to SEQ ID NO: 8 or a functional variant thereof. Preferably, a CTSS functional variant may comprise a nucleic acid sequence having at least 60%, more preferably 70%, 80% or 90% sequence identity with the sequence of SEQ ID NO:8. Preferably, a functional variant is a variant which expression is altered if the method according to the present invention is performed with a sample obtained from an individual having acute tuberculosis. The alteration of expression is preferably above a certain threshold, more preferably above 1.1, as described in the Examples.

In RT-qPCR any suitable primer that specifically binds to nucleic acid molecules of IL-19 may be used for detecting IL-19. Preferably, in addition to the primers a probe that specifically binds to nucleic acid molecules of IL-19 is used. For example, commercial Primer probe ThermoFisher (exon 6/7 boundary)=Hs00175407_m1 may be used.

The marker ncTRIM69 refers to processed, possibly non-coding, transcripts of the Tripartite motif containing 69 gene locus. Preferably, said transcripts are encoded by a nucleic acid molecule comprising a nucleic acid sequence according to SEQ ID NO: 9, 10 or 11 or a functional variant thereof. Preferably a functional variant of ncTRIM69 comprises a nucleic acid sequence having at least 70%, more preferably 75%, 80%, 85%, 90% or 95% sequence identity to SEQ ID NO: 9, 10 or 11. Preferably, a functional variant is a variant which expression is altered if the method according to the present invention is performed with a sample obtained from an individual having acute tuberculosis. The alteration of expression is preferably above a certain threshold, more preferably above 1.1, as described in the Examples.

In RT-qPCR any suitable primer that specifically binds to nucleic acid molecules of ncTRIM69 may be used for detecting ncTRIM69. Examples for suitable primers are nucleotides comprising a sequence according to SEQ ID NO: 12, 13, 14 and 15. Preferably, a primer pair comprising a nucleic acid sequence according to SEQ ID NO: 12 and SEQ ID NO: 13 or a primer pair comprising a nucleic acid sequence according to SEQ ID NO: 14 and SEQ ID NO: 15 is used. Preferably, in addition to the primers a probe that specifically binds to nucleic acid molecules of ncTRIM69 is used. For example a nucleic acid sequence comprising a sequence according to SEQ ID NO: 16 or 17 may be used as a probe. Said probes may comprise a fluorescence dye such as the 5′ Fluorophore FAM and/or a quencher such as BHQ1.

In an further embodiment the present invention provides a kit for performing a method according to the present invention, which kit comprises at least one antigen, at least two primer pairs for amplification of the at least two markers and preferably at least two probes for detecting the at least two markers. Preferably, the kit according to the present invention comprises at least two antigens.

In addition, the kit may comprise further components such as stimulants (antigens, positive and negative control stimulants), materials to perform cell-lysis (erythozyte-lysis buffer, PaxGene tubes) and RNA purification (lysis buffer, DNase, proteinase K, RNA-binding systems (bead-based, columns), washing buffer, elution buffers, materials for cDNA synthesis (e.g. gDNA wipeout buffer, reverse transcriptase, RT buffer, primer mix for RT (oligo-dT and random primers; or gene specific primers), dNTPs, RNaseH, 1-step RT-PCR enzyme mix (RT/Taq-Pol)), materials to perform qPCR (PCR buffer system (TaqMan Fast Universal PCR Master Mix, Reference gene Assay (TaqMan Gene Expression Assay RPLP0), primers & probes (for all markers), dNTPs, extraction control (internal control) like phage RNA, PCR control (e.g. plasmid), DNA Polymerase for PCR (Taq), Nucleotides, PCR plate (MicroAmp Fast Optical 96-Well reaction plate), PCR plate sealing (MicroAmp Optical Adhesive Film)), DNA ligase, adapter oligonucleotides, adapter-specific PCR primers, gene-specific capture oligonucleotides coupled to affinity tag (magnetic beads, biotin-streptavidin beads). Beyond that a kit may contain or reference, or contain parts of the following products NEBNext Ultra RNA Library Prep Kit for Illumina (New England Biolabs, USA) (catalog #E7530), NEBNext Poly(A) mRNA Magnetic Isolation module (catalog #E7490), KAPA library quantification kit (Kapa Biosystems, catalog #KK4824).

In a further preferred embodiment the kit comprises furthermore a pair of primers for amplification of the reference gene. Furthermore, it is according to the invention preferred if the kit contains additionally probes as well as a cell culture media.

In a further preferred embodiment according to the invention the kit additionally comprises RNA-stabilising reagents, a RT-master mix, a qPCR-master mix, a positive control, and a positive reagent. According to the invention a “positive control” is understood to be a defined amount of the marker DNA to be amplified. According to the invention a “positive reagent” is understood to be a reagent, which stimulates the marker of the blood cells, in particular APC and T cells unspecifically. Inventive examples for a “positive reagent” are PMA/Ionomycin. Preferably the RTT TB assay is controlled for cell functionality by an extra approach stimulating cells with a mixture of PMA (phorbol 12-myristate-13-acetate) and Ionomycin. Alternatively to PHA (phytohaemagglutinin) also SEB (staphylococcus enterotoxin B) and WGA (wheat germ agglutinin) can be used. Beyond that preferably stimulatory antibodies can be utilized alone or in combination (anti-CD-3; anti-CD40; anti-CD28, anti-CD49d). Beyond that preferably stimulatory pools of peptide like CEF pool can be utilized for control of cell functionality. For different marker combinations positive control reagents can be applied in single stimulations or in a combined stimulation.

In a further embodiment the present invention refers to the use of the marker ncTRIM69, which is encoded by a nucleic acid molecule comprising a nucleic acid sequence according to SEQ ID NO: 9, 10 or 11 or a functional variant thereof having at least 70%, more preferably 75%, 80%, 85%, 90% or 95% sequence identity to a nucleic acid sequence according to SEQ ID NO: 9, 10 or 11, in an in vitro method of diagnosing tuberculosis, in particular in an in vitro method of detecting infection with pathogens causing tuberculosis.

In a further embodiment the present invention refers to the use of a primer for ncTRIM69 as defined above and/or a probe for ncTRIM69 as defined above in an in vitro method of diagnosing tuberculosis, in particular in an in vitro method of detecting infection with pathogens causing tuberculosis, more particularly in an in vitro method for differentiating individuals being infected with pathogens causing tuberculosis and individuals being uninfected with pathogens causing tuberculosis, wherein individuals being infected with pathogens causing tuberculosis comprise individuals having a latent infection and individuals with active tuberculosis.

In a further embodiment the present invention provides a marker ncTRIM69 as defined above and/or a primer for ncTRIM69 as defined above or a probe for ncTRIM69 as defined above for use in a diagnostic method practised on the human or animal body for diagnosing tuberculosis, in particular for detecting infection with pathogens causing tuberculosis.

In still a further embodiment the present invention provides a kit for performing the TRIM-method as defined above comprising at least one antigen and at least one primer pair for amplification of the marker ncTRIM69 as described above, and preferably at least one probes for detecting the marker ncTRIM69 as described above. Preferably, the kit for the TRIM-method may comprise the additional kit components as described above.

In the following the invention is illustrated by the subsequent examples. These examples are to be considered as specific embodiments of the invention and shall not be considered to be limiting.

### Example 1—Sample Preparation, Stimulation and RNA Isolation—Manual System (Whole Blood/BMCs)

Stimulation of whole blood samples with TB proteins CFP10 and ESAT6 Blood was drawn from donors using sodium heparin monovettes. Until further use the blood was stored between 18-25° C. for no longer than 8 hours. The following steps were performed under sterile conditions in a class II biosafety laminar flow cabinet.

Blood samples from one donor were pooled and then 3 ml aliquots were made. Aliquots were either stimulated with 10 μg/ml CFP10 and 10 μg/ml ESAT6 or for the unstimulated control an equal volume of PBS was added. Additionally, as a positive control for stimulation, one blood aliquot was stimulated with 1 μg/ml PMA/Ionomycin. Samples were carefully mixed and afterwards incubated for 6 h at 37° C. and 5% CO₂. After incubation 5 volumes (15 ml) of buffer EL (QIAGEN—Cat No. 79217) were added and samples were incubated on ice for 15 min with two steps of vortexing in-between. Samples were then centrifuged for 10 min at 400 g and 4° C. The pellets was resuspended in 2 volumes (6 ml) of buffer EL and again centrifuged for 10 min at 400 g and 4° C. To each pellet 1.2 ml of lysis buffer (QIAGEN Buffer RLT (Cat No. 79216) with 40 mM DTT) were added and resuspended by pipetting 20 times. Samples were then immediately frozen in liquid nitrogen and stored at −80° C. until further use.

Stimulation of PBNICs with TB Proteins CFP10 and ESAT6

Blood was drawn from donors using sodium heparin monovettes. Until further use the blood was stored between 18-25° C. for no longer than 8 hours. The following steps were performed under sterile conditions in a class II biosafety laminar flow cabinet.

Blood was diluted with PBS in a 1:2 (blood to PBS) ratio. In a 50 ml centrifugation tube 15 ml Pancoll (PAN Biotech, Cat No. P04-60500) were added. Then 30 ml of the diluted blood was used to overlay the Pancoll. The tubes were centrifuged at 880 g for 30 min at room temperature with deactivated active breaking of the centrifuge.

The opaque-white PBMC layer was transferred to a new 50 ml centrifugation tube and filled up with PBS. The cells were centrifuged at 300 g for 10 min at room temperature. The pellet was resuspended in 1 ml PBS and transferred into a new 50 ml centrifugation tube, filled up with PBS, and again centrifuged at 300 g for 10 min at room temperature. The cell pellet was resuspended in 1 ml cell culture media. Cells were counted using a hemocytometer and diluted in cell culture media to a concentration of 2×10⁶ cells/ml. 2.5 ml aliquots were made and either stimulated with 10 μg/ml CFP10 and 10 μg/ml ESAT6 or for the unstimulated control an equal volume of PBS was added. Additionally, as a positive control, one blood aliquot was stimulated with 1 μg/ml PMA/Ionomycin.

Samples were carefully mixed and afterwards incubated for 6 h at 37° C. and 5% CO₂. After incubation cells were centrifuged for 10 min at 300 g at room temperature. To each pellet 600 μl of lysis buffer (QIAGEN Buffer RLT with 40 mM DTT) were added and resuspended by pipetting 20 times. Samples were then immediately frozen in liquid nitrogen and stored at −80° C. until further use.

RNA Isolation Using the RNeasy Mini Kit (QIAGEN)

For isolation of RNA from the frozen PBMCs or whole blood lysates (in Buffer RLT with 40 mM DTT) the RNeasy mini kit was used. Isolation was performed according to the QIAGEN manual. Elution was performed with 40 μl RNase-free water for PBMC samples or 25 μl RNase-free water for whole blood samples. RNA concentrations were determined by spectrophotometric analysis on a Nanodrop 1000 instrument.

### Example 2—Sample Preparation, Stimulation and RNA Isolation—Automated System (Whole Blood)

Stimulation of Whole Blood Samples with TB Proteins CFP10 and ESAT6

Blood was drawn from donors using sodium heparin monovettes. Until further use the blood was stored between 18-25° C. for no longer than 8 hours. The following steps were performed under sterile conditions in a class II biosafety laminar flow cabinet.

Blood samples from one donor were pooled and then 2.5 ml aliquots were made. Aliquots were either stimulated with 10 μg/ml CFP10 and 10 μg/ml ESAT6 or for the unstimulated control an equal volume of PBS was added. Additionally, as a positive control for stimulation, one blood aliquot was stimulated with 1 μg/ml PMA/Ionomycin. Samples were carefully mixed and afterwards incubated for 6 h at 37° C. and 5% CO₂. After incubation the complete 2.5 ml of each aliquot were transferred to a separate PAXgene Blood RNA tube (QIAGEN—Cat No. 762125) and mixed by inverting the tube 10 times. The PAXgene Blood RNA tubes were incubated for 16-24 h at room temperature according to the distributor's instructions and afterwards stored at −20° C. until further use.

RNA Isolation Using the MagNA Pure 96 System (Roche)

PAXgene Blood RNA tubes were thawed at room temperature for 2 h and afterwards centrifuged at 4000 g for 10 min at room temperature. The pellet was resuspended in 4 ml RNase-free water by vortexing and again centrifuged at 4000 g for 10 min at room temperature. The pellet was dissolved in 400 μl RNase-free PBS by vortexing.

For RNA isolation a MagNA Pure 96 instrument (Roche—Cat No. 06541089001) and the “MagNA Pure 96 Cellular RNA Large Volume Kit” (Roche—Cat No. 05467535001) was used. Either 400 μl or 200 μl of each dissolved “PAXgene Blood RNA tube” pellet were transferred into one well of a MagNA Pure 96 Processing Cartridge and the predefined “RNA PAXgene LV” or “RNA PAXgene Half Tube LV” MagNA Pure 96 protocols were run, respectively. Samples were eluted in 100 μl or 50 μl of the kit's elution buffer for the “RNA PAXgene LV” or “RNA PAXgene Half Tube LV” protocols, respectively.

RNA concentrations were determined by spectrophotometric Analysis on a NanoDrop 1000 instrument.

cDNA Synthesis

For cDNA synthesis the “QuantiTect Reverse Transcription Kit” (QIAGEN—Cat No. 205313) was used.

In short, in a first step to eliminate gDNA, 1 μg of RNA was mixed with 41 gDNA Wipeout Buffer (7×) in an overall 14 μl reaction volume with RNase-free water. Reaction was incubated at 42° C. for 2 min and afterwards immediately put on ice. Then 4 μl Quantiscript RT Buffer (5×), 1 μl RT Primer Mix and 1 μl Quantiscript Reverse Transcriptase were added, mixed, and incubated at 42° C. for 30 min. Afterwards the RT reaction was stopped by heat-inactivating the Quantiscript Reverse Transcriptase at 95° C. for 3 min.

### Example 3-qPCR to Determine mRNA Levels of Marker-Genes

For each qPCR reaction 1 μl of reverse transcribed cDNA as obtained in Example 2 was used and mixed with 5 μl of TaqMan Fast Universal Master Mix (Thermo Fisher—Cat. No 4366073), 0.3 μl of gene-specific forward and reverse primer (10 μM stock concentration, final concentration 300 nM each), 0.2 μl of a gene-specific fluorescent probe (10 μM stock concentration, final concentration 200 nM), 0.167 μl of a 60×RPLP0 TaqMan® Gene Expression Assay (Thermo Fisher—Cat No. 4331182—Assay ID: Hs99999902_m1), and 3.033 μl of water.

For detection of indicated makers following primers/probes or commercial assays have been used:
IFNG:
forward primer according to SEQ ID NO: 2
reverse primer according to SEQ ID NO: 3

probe:
BoTMR-TTCATGTATTGCTTTGCGTTGGACATTCAA-BBQ

ncTRIM69:
forward primer according to SEQ ID NO: 12
reverse primer according to SEQ ID NO: 13

probe:
6FAM-CCGGGAAAGTGGCACACTCCTGG-BHQ1

CTSS: ThermoFisher Taqman Assay Hs00175407_m1 (Cat No. 4331182)
IL19: ThermoFisher Taqman Assay Hs00604657_m1 (Cat No. 4331182)
GBP5: ThermoFisher Taqman Assay Hs00369472_m1 (Cat No. 4331182)
CXCL10: ThermoFisher Taqman Assay Hs00171042_m1 (Cat No. 4331182)

PCR was run either on a StepOnePlus (Thermo Fisher—Cat No.—4376600) or QuantStudio 3 (Thermo Fisher—Cat No. A28136) Real-Time PCR system. The two-step PCR-protocol starts with an initial 95° C. denaturation step for 20 sec and then completes 40 cycles of 95° C. for 3 sec and subsequent 60° for 30 sec with data collection during the later. Thresholds for Ct values were set manually after the run and the Ct values were then exported for data analysis.

### Example 4—Data Analysis and Fold Change Calculations

For data analysis Ct mean values for replicates of marker gene and RPLP0 samples were used. The DNA quantity (D) of marker genes and RPLP0 was calculated using the Ct values (Ct) and the PCR efficiency (e) of each PCR reaction, using the following formula:

D=Ce

Normalized DNA quantity for marker genes (Nₘ) was calculated using the DNA quantity of marker genes (Dₘ) and the DNA quantity of the housekeeping gene RPLP0 (Dₕ) in the same samples, using the following formula:

Nₘ=Dₘ/Dₕ

For expression fold change calculations of each marker gene (fcₘ) through stimulation the normalized DNA quantities from the stimulated (Nₘ(S)) and the unstimulated (Nₘ(U)) samples from each donor obtained from Example 1 and 2 were used in the following formula:

fcₘ=(Nₘ(S))/(Nₘ(U))

Fold change values were used to classify donors as TB-infected or -uninfected using the previously designed Classifier (random forest approach) as e.g. exemplified in examples 6 and 7.

### Example 5: Threshold Analysis of mRNA Fold-Changes Between Unstimulated and with ESAT-6/CFP-10 Stimulated Whole Blood Samples of Marker Genes CXCL10, GBP5, and IFNG to Identify TB Infected Individuals

To design a method to decide, if an individual is infected with tuberculosis, mRNA expression differences, determined by RT-qPCR, between unstimulated and with TB-antigens stimulated whole blood samples from individuals with known TB status were analyzed.

For this purpose blood was drawn from a collective of 27 not TB infected persons, 30 latent TB infected (LTBI) persons, and 30 individuals with active TB (ATB). Whole blood samples were then stimulated with CFP10 and ESAT6, and RNA was isolated as described in example 1. The isolated RNA was used for cDNA synthesis and qPCR analysis as described in the previous examples. For all stimulated or unstimulated samples qPCRs on marker-genes CXCL10. GBP5, and IFNG, as well as on the housekeeping gene RPLP0 were performed RPLP0 was used to normalize marker-gene expression and differences between stimulated and unstimulated samples from one donor was used to calculate the fold change as described in example 4.

To discriminate between not TB infected and TB infected persons thresholds for the fold changes of each marker gene were defined. ATB and LTBI were not differentiated and both defined as infected individuals.

The fold change threshold for CXCL10 was set at 3.2, for GPB5 at 1.11, and for IFNG at 5. Since all three maker genes were upregulated in TB infected compared to not-infected individuals, values above the threshold were used as indications of a TB infection. For example, using only the marker gene IFNG fold changes above 6.5 would result in a classification as TB infected. A fold change of 6.5 and below again would result in a classification as not-infected with TB.

Latent donor 66 (LD66) as an example has an IFNG fold change of 7.74 in the stimulated and unstimulated whole blood sample and would therefore result in a correct classification as TB infected. Healthy donor 55 on the other hand has an IFNG fold change of 1.02 and was hence correctly classified as not TB infected.

To improve predictions of the infection status of patients, all possible combination of two markers and the combination of all three markers were tested.

For the combination of two markers at once two different analyses were performed: (i) at least one marker has to be above threshold for classification as infected. Not-infected individuals are in this case defined by fold changes of both markers below the defined threshold. All other individuals with one or both marker's fold changes above threshold are classified as TB infected. (ii) Both markers have to be above threshold for classification as infected. If one or both marker are below threshold the individual would be classified as not-infected.

Latent donor 67 with an IFNG fold change of 2.73 for example would have been classified incorrect as not infected, if only IFNG would be considered. However this donor has a CXCL10 fold change of 38.21 and the combined analysis of IFNG and CXCL10 with as in (i) described at least one marker above threshold results in the correct classification as an individual with TB infection.

Accordingly for the combination of all three markers at once three different analyses were performed: fold changes of (i) at least one marker, (ii) at least two markers, or (iii) all markers have to be above threshold for classification as infected.

All possible combinations of genes were tested in this way and compared to the results of obtained by single gene threshold analysis. As quality determining criterion the sum of sensitivity and specificity for identifying the correct TB infection status in the tested collective (27 not-infected and 60 infected persons) was calculated.

As shown in Table 1, the combination of CXCL10 and INFG, under the condition that both their fold changes have to be above threshold, results in an improved combined sensitivity and specificity compared to their single marker analysis. Also the combination of CXCL10 and GBP5 are improved using the condition that both markers have to be above the threshold.

By combining all three tested marker under the condition that at least two of the three have to be above threshold for classification as TB infected the score for combined sensitivity and specificity could be further improved and patient can be better categorized.

Active donor 62 for example has a CXCL10 fold change of 2.6, GBP5 fold change of 1.2, and an IFNG fold change of 6.14. With the preferred 2 gene analysis of CXCL10 and GPB5 with the condition that both have to be above threshold for classification of infected, this individual would have been incorrectly labeled as not-infected. However, in the three gene analysis, additionally including IFNG, and the condition that at least two markers have to be above threshold for classification as infected with TB, this individual is labeled correctly as TB infected.

TABLE 1
Sensitivities and specificities of different marker
combinations determined by threshold analysis.

| | | No. of genes at least needed | | | |
|---|---|---|---|---|---|
| Marker gene | No. of | above threshold for | | | |
| combinations | genes | classification as infected | Sensitivity | Specificity | Sens + Spec |
| CXCL10 | 1 | 1 | 88.33 | 88.89 | 1.772 |
| GBP5 | 1 | 1 | 90.00 | 62.96 | 1.530 |
| IFNG | 1 | 1 | 78.33 | 100.00 | 1.783 |
| CXCL10/GPB5 | 2 | 1 | 95.00 | 51.85 | 1.469 |
| CXCL10/IFNG | 2 | 1 | 90.00 | 88.89 | 1.789 |
| GBP5/IFNG | 2 | 1 | 95.00 | 62.96 | 1.580 |
| CXCL10/GPB5 | 2 | 2 | 83.33 | 100.00 | 1.833 |
| CXCL10/IFNG | 2 | 2 | 76.67 | 100.00 | 1.767 |
| GBP5/IFNG | 2 | 2 | 73.33 | 100.00 | 1.733 |
| CXCL10/GPB5/ | 3 | 1 | 95.00 | 51.85 | 1.469 |
| IFNG | | | | | |
| CXCL10/GPB5/ | 3 | 2 | 90.00 | 100.00 | 1.900 |
| IFNG | | | | | |
| CXCL10/GPB5/ | 3 | 3 | 71.67 | 100.00 | 1.717 |
| IFNG | | | | | |

### Example 6: Infection Detection from Whole Blood Using Random-Forest Classifiyer

For the Random Forest classifier analyses, two patient collectives were built: a training collective of approximately 90 patients (including ˜30 healthy, ˜30 latently-infected and ˜30 actively-infected donors) for the classifier generation, and a test collective of approximately 60 patients (including ˜20 healthy, ˜20 latently-infected and ˜20 actively-infected donors) for the classifier validation.

Each collective was built based on the following criteria. Healthy donors were symptom-free healthy volunteers. Latent TB donors were symptom-free and either IGRA-positive or classified based on clinician's decision (LD38, LD40, LD73 and LD75). Active TB donors were patients with symptoms suspicious for tuberculosis and who were later confirmed as actively-infected with M. tuberculosis using at least one of the following method, applied on collected clinical specimens (e.g., sputum, urine, cerebrospinal fluid, or biopsy): direct AFB smear microscopy, direct detection of pathogen by nucleic acid amplification (PCR), and/or specimen culturing.

In case of the following donors, confirmatory diagnostics like IGRA, culture, PCR and/or microscopy were not yet available at the time of the experiment: LD81, LD85, LD86, LD89, AD 91, AD92, AD93, AD96, AD100.

Results of gene expression analysis in each individual are expressed as fold-change (antigen-stimulated over unstimulated condition) and shown in the respective tables (Table 4B, 5B, 8, 9).

### Definitions and Abbreviations

TP: true positive
TN: true negative
FP: false positive
FN: false negative
TPR (true positive rate)=TP/(TP+FN)=sensitivity
TNR (true negative rate)=TN/(TN+FP)=specificity
FPR (false positive rate)=1−TNR
Accuracy=(TP+TN)/Total population, where Total population=TP+TN+FP+FN
AUC=Area under the curve=Integral over the graph that results from computing TPR (sensitivity) and FPR (1—specificity) for many different thresholds
X.recall=Percentage of correctly classified observations in the class X=Percentage of observations from class X classified as class X

Thus, in the performance table below, “infected.recall” refers to the % of infected patients correctly classified as infected (also defined as sensitivity or TPR), and “noninfected.recall” refers to the % of non-infected subjects correctly classified as non-infected (also defined as specificity or TNR).

The aim of this study was to establish classifiers for preselected marker combinations enabling a robust identification of individuals infected with tuberculosis pathogens. In this experiments anticoagulated whole blood samples of 27 healthy (no previous contact with tuberculosis pathogens), 30 latently-infected and 30 actively-infected donors (training samples) were stimulated with ESAT6 and CFP10 antigens as essentially described in example 1 (paragraph “stimulation of whole blood samples). In this experiment, patients infected with pathogens causing tuberculosis were preselected with regard to substantial IFNG secretion from isolated PBMC upon stimulation with ESAT6/CFP10 proteins and thus patient collective was biased for the marker IFNG.

RNA isolation was performed as described in example 1. QPCR was performed as described in example 3. Then, random-forest classifiers were established using the software R [3.5.0] in combination with the packages ranger [0.9.0], readxl [1.1.0], stringr [1.3.0] and mlr [2.12.1]. The measurements of the samples described in Table 4A/B (training samples; N=87, including 27 healthy, 30 latently-infected and 30 actively-infected donors) were log 2-transformed. Afterwards, the function ranger( ) was used for training with the following parameters: number of trees=1e3, minimal node size=5, split rule=“extratrees” with the number of random splits set to 5 and the number of variables to possibly split at set to 1. On these training samples, the random forest resulted in performances shown in Table 2. Considering a scoring based on the sum of sensitivity and specificity (last column), performances ranged from a score of 1.7372 for IFNG alone to a score of 1.8636 for CXCL10/GBP5/IFNG. The performance of IFNG alone (sensitivity: 88.73%; specificity: 84.99%; score sensitivity+specificity: 1.7372) was improved by the addition of one additional marker (GBP5/IFNG; sensitivity: 89.6%; specificity: 85.24%; score: 1.7484) or of two additional markers (CXCL10/GBP5/IFNG; sensitivity: 92.27%; specificity: 94.09%; score: 1.8636) (Table 2).

Established classifiers were independently validated with RNA samples, obtained from specifically stimulated anticoagulated whole blood of 23 healthy, 20 latently-infected and 20 actively-infected donors (Table 5A/B); which have been generated as described before for the training cohort. The participants of this study were not preselected regarding levels of IFNG production and thus constitute a representative collective of tuberculosis patients.

Herein, performances of preselected marker combinations (shown in Table 3) ranged from a score (sensitivity+specificity) of 1.7565 for IFNG alone to 1.8565 for CXCL10/GBP5/IFNG/ncTRIM69. On this validation set, the performance of GBP5 alone (sensitivity: 92.50%; specificity: 86.96%; score sensitivity+specificity: 1.7946) was improved by the addition of two additional markers (CXCL10/GBP5/IFNG; sensitivity: 90.00%; specificity: 91.30%; score: 1.8130) or of three additional markers (CXCL10/GBP5/IFNG/ncTRIM69; sensitivity: 90.00%; specificity: 95.65%; score: 1.8565) (Table 3). Thus, established classifiers for described marker combinations allow a robust identification of patients infected by tuberculosis pathogens.

TABLE 2
Classifier training set (27 non-infected/30 latent TB/30 active TB; N = 87)

| | | | | | Scoring: |
|---|---|---|---|---|---|
| | | infected.recall | noninfected.recall | | sum |
| Genes | Accuracy | (sensitivity) | (specificity) | AUC | sens + spec |
| CXCL10/GBP5/IFNG | 0.9283 | 0.9227 | 0.9409 | 0.9709 | 1.8636 |
| CXCL10/GPB5/IFNG/ncTRIM69 | 0.9226 | 0.9213 | 0.9253 | 0.9739 | 1.8467 |
| CXCL10/GPB5/IFNG/IL19/ | 0.9197 | 0.9203 | 0.9193 | 0.9679 | 1.8397 |
| ncTRIM69 | | | | | |
| CTSS/CXCL10/GBP5/IFNG | 0.9197 | 0.9233 | 0.9125 | 0.9650 | 1.8359 |
| CXCL10/IFNG | 0.9113 | 0.9083 | 0.9171 | 0.9577 | 1.8254 |
| CTSS/CXCL10/IFNG | 0.9075 | 0.9023 | 0.9208 | 0.9556 | 1.8231 |
| CXCL10/GBP5/IFNG/IL19 | 0.9141 | 0.9190 | 0.9036 | 0.9669 | 1.8226 |
| CTSS/CXCL10/GBP5/IFNG/ | 0.9132 | 0.9193 | 0.8999 | 0.9681 | 1.8192 |
| ncTRIM69 | | | | | |
| CXCL10/IFNG/ncTRIM69 | 0.9082 | 0.9070 | 0.9108 | 0.9618 | 1.8178 |
| CXCL10/IFNG/IL19 | 0.9070 | 0.9093 | 0.9021 | 0.9562 | 1.8115 |
| CXCL10/IFNG/IL19/ncTRIM69 | 0.9069 | 0.9083 | 0.9025 | 0.9587 | 1.8109 |
| CTSS/CXCL10/IFNG/ncTRIM69 | 0.9035 | 0.9103 | 0.8903 | 0.9615 | 1.8006 |
| CXCL10/GPB5/ncTRIM69 | 0.9025 | 0.9120 | 0.8805 | 0.9640 | 1.7925 |
| CTSS/CXCL10/IFNG/IL19/ | 0.9009 | 0.9167 | 0.8685 | 0.9607 | 1.7852 |
| ncTRIM69 | | | | | |
| CTSS/CXCL10/IFNG/IL19 | 0.8946 | 0.9030 | 0.8791 | 0.9573 | 1.7821 |
| CTSS/CXCL10/GBP5/IFNG/IL19 | 0.8967 | 0.9107 | 0.8680 | 0.9612 | 1.7787 |
| CTSS/CXCL10/GBP5/IFNG/IL19/ | 0.8935 | 0.9140 | 0.8497 | 0.9641 | 1.7637 |
| ncTRIM69 | | | | | |
| CTSS/CXCL10/GPB5/ncTRIM69 | 0.8858 | 0.8993 | 0.8571 | 0.9575 | 1.7564 |
| CXCL10/ncTRIM69 | 0.8853 | 0.8993 | 0.8540 | 0.9530 | 1.7533 |
| CXCL10/GBP5 | 0.8839 | 0.8947 | 0.8580 | 0.9557 | 1.7527 |
| CXCL10/IL19/ncTRIM69 | 0.8794 | 0.8873 | 0.8620 | 0.9552 | 1.7493 |
| GBP5/IFNG | 0.8823 | 0.8960 | 0.8524 | 0.9594 | 1.7484 |
| IFNG/ncTRIM69 | 0.8813 | 0.8947 | 0.8535 | 0.9485 | 1.7481 |
| CXCL10/GBP5/IL19/ncTRIM69 | 0.8809 | 0.8920 | 0.8556 | 0.9587 | 1.7476 |
| CTSS/CXCL10/GBP5 | 0.8810 | 0.8987 | 0.8432 | 0.9419 | 1.7419 |
| GBP5/IFNG/ncTRIM69 | 0.8810 | 0.8990 | 0.8427 | 0.9627 | 1.7417 |
| CTSS/GBP5/IFNG | 0.8801 | 0.9020 | 0.8364 | 0.9541 | 1.7384 |
| IFNG | 0.8753 | 0.8873 | 0.8499 | 0.9312 | 1.7372 |

TABLE 3
Classifier test set (23 non-infected/20 latent TB/20 active TB; N = 63)

| | | | | | scoring: |
|---|---|---|---|---|---|
| | | infected.recall | noninfected.recall | | sum |
| Genes | Accuracy | (sensitivity) | (specificity) | AUC | sens + spec |
| CXCL10/GBP5/IFNG/ncTRIM69 | 0.9206 | 0.9000 | 0.9565 | 0.9489 | 1.8565 |
| CTSS/CXCLIO/GBP5/IFNG/ncTRIM69 | 0.9206 | 0.9000 | 0.9565 | 0.9554 | 1.8565 |
| CXCL10/GPB5/IFNG/IL19/ncTRIM69 | 0.9206 | 0.9000 | 0.9565 | 0.9424 | 1.8565 |
| CTSS/CXCL10/GBP5/IFNG/IL19/ | 0.9206 | 0.9000 | 0.9565 | 0.9522 | 1.8565 |
| ncTRIM69 | | | | | |
| GBP5/IFNG/IL19 | 0.9048 | 0.8750 | 0.9565 | 0.9587 | 1.8315 |
| GPB5/IFNG/ncTRIM69 | 0.9048 | 0.8750 | 0.9565 | 0.9446 | 1.8315 |
| CTSS/GBP5/IFNG/ncTRIM69 | 0.9048 | 0.8750 | 0.9565 | 0.9576 | 1.8315 |
| GPB5/IFNG/IL19/ncTRIM69 | 0.9048 | 0.8750 | 0.9565 | 0.9500 | 1.8315 |
| CTSS/GPB5/IFNG/IL19/ncTRIM69 | 0.9048 | 0.8750 | 0.9565 | 0.9652 | 1.8315 |
| CXCL10/GPB5/IFNG | 0.9048 | 0.9000 | 0.9130 | 0.9522 | 1.8130 |
| CTSS/CXCL10/GBP5/IFNG | 0.9048 | 0.9000 | 0.9130 | 0.9620 | 1.8130 |
| CTSS/CXCLIO/GBP5/ncTRIM69 | 0.9048 | 0.9000 | 0.9130 | 0.9478 | 1.8130 |
| CXCL10/GBP5/IFNG/IL19 | 0.9048 | 0.9000 | 0.9130 | 0.9424 | 1.8130 |
| CTSS/CXCLIO/GPB5/IL19/ncTRIM69 | 0.9048 | 0.9000 | 0.9130 | 0.9359 | 1.8130 |
| GBP5 | 0.9048 | 0.9250 | 0.8696 | 0.9402 | 1.7946 |
| GBP5/IFNG | 0.8889 | 0.8750 | 0.9130 | 0.9533 | 1.7880 |
| CTSS/CXCL10/GPB5 | 0.8889 | 0.8750 | 0.9130 | 0.9500 | 1.7880 |
| CTSS/GBP5/IFNG | 0.8889 | 0.8750 | 0.9130 | 0.9663 | 1.7880 |
| CXCL10/GPB5/IL19 | 0.8889 | 0.8750 | 0.9130 | 0.9250 | 1.7880 |
| CXCL10/IFNG/IL19 | 0.8889 | 0.8750 | 0.9130 | 0.9391 | 1.7880 |
| CXCL10/IFNG/ncTRIM69 | 0.8889 | 0.8750 | 0.9130 | 0.9315 | 1.7880 |
| CTSS/CXCL10/IFNG/ncTRIM69 | 0.8889 | 0.8750 | 0.9130 | 0.9402 | 1.7880 |
| CTSS/GBP5/IFNG/IL19 | 0.8889 | 0.8750 | 0.9130 | 0.9674 | 1.7880 |
| CXCL10/GBP5/IL19/ncTRIM69 | 0.8889 | 0.8750 | 0.9130 | 0.9283 | 1.7880 |
| CXCL10/IFNG/IL19/ncTRIM69 | 0.8889 | 0.8750 | 0.9130 | 0.9391 | 1.7880 |
| CTSS/CXCL10/IFNG/IL19/ncTRIM69 | 0.8889 | 0.8750 | 0.9130 | 0.9391 | 1.7880 |
| CTSS/CXCL10/GBP5/IFNG/IL19 | 0.8889 | 0.9000 | 0.8696 | 0.9576 | 1.7696 |
| CTSS/GPB5 | 0.8730 | 0.8500 | 0.9130 | 0.9413 | 1.7630 |
| CXCL10/GPB5 | 0.8730 | 0.8500 | 0.9130 | 0.9500 | 1.7630 |
| CTSS/GBP5/IL19 | 0.8730 | 0.8500 | 0.9130 | 0.9141 | 1.7630 |
| CXCL10/GBP5/ncTRIM69 | 0.8730 | 0.8500 | 0.9130 | 0.9413 | 1.7630 |
| IFNG | 0.8571 | 0.8000 | 0.9565 | 0.9424 | 1.7565 |

TABLE 4A
(training samples; N = 87)

| | Confirmed | | | | | | |
|---|---|---|---|---|---|---|---|
| | Diagnosis | | | IGRA | | Biopsy/ | |
| Patient | TB Active/ | | BCG | (QFN/ | | Microscopy | Culture |
| ID | Latent | Diagnose TB | Vaccinated | T-Spot) | PCR | Findings | Results |
| HD28 | healthy | not infected | no | n.d. | — | — | — |
| HD29 | healthy | not infected | no | n.d. | — | — | — |
| HD30 | healthy | not infected | no | n.d. | — | — | — |
| HD40 | healthy | not infected | unknown | negative | — | — | — |
| HD41 | healthy | not infected | — | negative | negative | — | — |
| HD42 | healthy | not infected | unknown | negative | — | — | — |
| HD43 | healthy | not infected | no | negative | — | — | — |
| HD44 | healthy | not infected | no | negative | — | — | — |
| HD47 | healthy | not infected | yes | negative | — | — | — |
| HD49 | healthy | not infected | yes | negative | — | — | — |
| HD50 | healthy | not infected | yes | negative | — | — | — |
| HD51 | healthy | not infected | yes | negative | — | — | — |
| HD52 | healthy | not infected | no | negative | — | — | — |
| HD53 | healthy | not infected | no | n.d. | n.d. | — | — |
| HD54 | healthy | not infected | unknown | n.d. | — | | — |
| HD55 | healthy | not infected | yes | negative | — | — | — |
| HD56 | healthy | not infected | unknown | n.d. | — | — | — |
| HD57 | healthy | not infected | no | n.d. | n.d. | — | — |
| HD58 | healthy | not infected | unknown | negative | — | — | — |
| HD59 | healthy | not infected | unknown | negative | — | — | — |
| HD60 | healthy | not infected | unknown | n.d. | — | — | — |
| HD61 | healthy | not infected | unknown | n.d. | — | — | — |
| HD62 | healthy | not infected | yes | negative | — | — | — |
| HD64 | healthy | not infected | no | positive | — | — | — |
| HD65 | healthy | not infected | no | negative | — | — | — |
| HD66 | healthy | not infected | no | negative | — | — | — |
| HD67 | healthy | not infected | no | negative | | — | — |
| LD22 | latent | — | no | positive | n.d. | n.d. | n.d. |
| LD47 | latent | — | unknown | positive | n.d. | — | negative |
| LD48 | latent | — | — | positive | n.d. | — | n.d. |
| LD49 | latent | — | unknown | positive | positive | — | positive |
| LD52 | latent | — | unknown | positive | n.d. | — | negative |
| LD53 | latent | — | unknown | positive | — | — | positive |
| LD54 | latent | — | unknown | positive | positive | — | negative |
| LD55 | latent | — | unknown | positive | negative | — | negative |
| LD56 | latent | — | unknown | positive | n.d. | — | negative |
| LD57 | latent | — | unknown | positive | n.d. | — | n.d. |
| LD58 | latent | — | yes | positive | n.d. | n.d. | n.d. |
| LD59 | latent | — | unknown | positive | negative | — | negative |
| LD60 | latent | — | — | positive | n.d. | — | n.d. |
| LD61 | latent | treated as | unknown | positive | positive | — | positive |
| | | active TB | | | | | |
| | | previously, | | | | | |
| | | treatment was | | | | | |
| | | ended 0.5 years | | | | | |
| | | ago | | | | | |
| LD62 | latent | — | unknown | positive | n.d. | — | negative |
| LD63 | latent | — | unknown | positive | negative | — | n.d. |
| LD65 | latent | — | unknown | positive | negative | — | negative |
| LD66 | latent | — | unknown | positive | negative | — | negative |
| LD67 | latent | — | unknown | positive | n.d. | — | n.d. |
| LD68 | latent | — | unknown | positive | n.d. | — | n.d. |
| LD69 | latent | — | yes | positive | n.d. | — | n.d. |
| LD70 | latent | — | yes | positive | n.d. | — | n.d. |
| LD71 | latent | — | yes | positive | n.d. | — | n.d. |
| LD72 | latent | — | unknown | positive | n.d. | — | n.d. |
| LD73 | latent | — | unknown | negative | negative | — | negative |
| LD74 | latent | — | unknown | positive | negative | — | negative |
| LD75 | latent | — | | inconclusive | — | — | |
| LD76 | latent | — | unknown | positive | negative | — | negative |
| LD77 | latent | — | no | positive | n.d. | — | n.d. |
| LD78 | latent | — | — | positive | — | — | — |
| AD22 | active | pulmonary | unknown | n.d. | positive | negative | positive |
| AD52 | active | pulmonary | unknown | positive | positive | — | positive |
| AD53 | active | — | unknown | n.d. | positive | — | positive |
| AD54 | active | — | unknown | positive | positive | — | positive |
| AD55 | active | pulmonary | unknown | positive | positive | — | positive |
| AD56 | active | extrapulmonary | unknown | positive | negative | positive | negative |
| AD57 | active | pulmonary | yes | positive | positive | n.d. | positive |
| AD58 | active | pulmonary | unknown | positive | positive | n.d. | positive |
| AD59 | active | — | unknown | positive | positive | negative | positive |
| AD60 | active | — | unknown | n.d. | positive | n.d. | positive |
| AD61 | active | pulmonary | unknown | positive | positive | negative | negative |
| AD62 | active | pulmonary | unknown | positive | negative | n.d. | positive |
| AD63 | active | pulmonary | unknown | positive | positive | n.d. | positive |
| AD64 | active | pulmonary | unknown | n.d. | positive | — | positive |
| AD66 | active | pulmonary | unknown | n.d. | positve | — | positive |
| AD67 | active | pulmonary | unknown | positive | positive | n.d. | positive |
| AD68 | active | pulmonary, | no | positive | positive | n.d. | positive |
| | | lymph nodes | | | | | |
| AD69 | active | pulmonary | unknown | positive | positive | n.d. | negative |
| AD70 | active | pulmonary, | — | positive | positive | — | positive |
| | | extrapulmonary | | | | | |
| AD71 | active | pulmonary | unknown | positive | positive | n.d. | positive |
| AD72 | active | pulmonary | unknown | negative | positive | n.d. | positive |
| AD73 | active | pulmonary | unknown | positive | positive | n.d. | positive |
| AD74 | active | pulmonary | unknown | n.d. | n.d. | — | negative |
| AD75 | active | pulmonary | unknown | positive | positive | — | positive |
| AD76 | active | Suspicion not | unknown | positive | — | — | negative |
| | | confirmed | | | | | |
| | | culturally | | | | | |
| AD77 | active | pulmonary | unknown | n.d. | n.d. | negative | positive |
| AD78 | active | pulmonary | unknown | positive | positive | — | positive |
| AD79 | active | pulmonary | unknown | n.d. | positve | n.d. | positive |
| AD80 | active | pulmonary | unknown | n.d. | positve | n.d. | positive |
| AD81 | active | pulmonary | unknown | n.d. | — | n.d. | positive |

TABLE 4B
(training samples; N = 87)

| | Fold | Fold | Fold | Fold | Fold | |
|---|---|---|---|---|---|---|
| Patient | change | change | change | change | change | Fold change |
| ID | (CTSS) | (CXCL10) | (GBP5) | (IFNG) | (IL19) | (ncTRIM69) |
| HD28 | 0.96 | 1.15 | 0.89 | 1.26 | 0.79 | 0.92 |
| HD29 | 0.81 | 0.89 | 0.86 | 1.57 | 0.51 | 0.99 |
| HD30 | 1.05 | 0.49 | 0.85 | 0.94 | 4.32 | 1.06 |
| HD40 | 0.75 | 1.01 | 0.72 | 3.53 | 1.01 | 0.78 |
| HD41 | 1.10 | 0.63 | 1.20 | 3.07 | 0.53 | 0.81 |
| HD42 | 0.72 | 1.45 | 0.85 | 1.46 | 0.24 | 0.79 |
| HD43 | 1.21 | 1.41 | 1.11 | 1.10 | 1.00 | 0.99 |
| HD44 | 0.94 | 0.94 | 0.90 | 1.73 | 0.11 | 0.87 |
| HD47 | 0.99 | 1.34 | 0.92 | 0.71 | 0.81 | 0.98 |
| HD49 | 0.90 | 1.02 | 1.00 | 1.56 | 0.98 | 0.70 |
| HD50 | 1.27 | 3.07 | 1.57 | 0.28 | 3.56 | 1.20 |
| HD51 | 0.94 | 1.83 | 0.90 | 1.18 | 0.71 | 1.09 |
| HD52 | 1.01 | 0.98 | 0.95 | 0.86 | 1.61 | 0.95 |
| HD53 | 0.73 | 3.76 | 0.76 | 1.15 | 0.57 | 0.90 |
| HD54 | 0.95 | 1.04 | 0.84 | 1.27 | 0.95 | 1.10 |
| HD55 | 0.92 | 3.61 | 0.98 | 1.02 | 1.44 | 0.58 |
| HD56 | 1.11 | 0.77 | 1.15 | 0.97 | 0.85 | 1.05 |
| HD57 | 1.20 | 2.57 | 1.23 | 1.95 | 1.04 | 1.90 |
| HD58 | 1.12 | 11.26 | 1.03 | 0.81 | 0.98 | 1.04 |
| HD59 | 1.12 | 0.72 | 1.12 | 0.80 | 1.25 | 1.21 |
| HD60 | 0.98 | 3.16 | 0.93 | 1.39 | 1.61 | 1.12 |
| HD61 | 0.93 | 2.75 | 1.36 | 2.19 | 1.61 | 1.18 |
| HD62 | 1.01 | 0.96 | 1.09 | 0.65 | 1.13 | 1.34 |
| HD64 | 1.27 | 0.98 | 1.25 | 1.40 | 1.11 | 0.64 |
| HD65 | 0.96 | 2.48 | 1.10 | 0.80 | 0.85 | 1.03 |
| HD66 | 1.20 | 1.16 | 1.33 | 2.29 | 0.92 | 1.09 |
| HD67 | 1.49 | 1.42 | 1.30 | 1.53 | 2.33 | 1.15 |
| LD22 | 0.97 | 160.95 | 1.39 | 2.36 | 0.33 | 1.06 |
| LD47 | 1.16 | 113.95 | 5.41 | 22.32 | 6.38 | 2.44 |
| LD48 | 0.94 | 58.10 | 1.09 | 7.92 | 1.18 | 1.18 |
| LD49 | 0.88 | 98.31 | 2.87 | 26.92 | 1.00 | 1.03 |
| LD52 | 1.06 | 359.32 | 5.52 | 16.94 | 1.82 | 1.47 |
| LD53 | 1.08 | 17.62 | 1.26 | 1.57 | 0.97 | 1.38 |
| LD54 | 1.24 | 59.56 | 6.52 | 289.08 | 2.24 | 1.63 |
| LD55 | 1.02 | 801.30 | 5.61 | 249.67 | 4.11 | 2.16 |
| LD56 | 1.20 | 1297.69 | 8.31 | 34.27 | 1.54 | 1.42 |
| LD57 | 1.56 | 146.97 | 4.57 | 3.63 | 1.52 | 1.65 |
| LD58 | 1.01 | 542.38 | 5.31 | 3.04 | 0.91 | 2.11 |
| LD59 | 1.13 | 1.34 | 0.96 | 0.97 | 1.99 | 0.67 |
| LD60 | 0.70 | 57.24 | 1.11 | 12.07 | 0.77 | 0.88 |
| LD61 | 1.00 | 9.38 | 1.52 | 1.14 | 0.88 | 1.01 |
| LD62 | 1.45 | 191.14 | 7.15 | 33.05 | 2.68 | 5.07 |
| LD63 | 1.02 | 0.95 | 0.95 | 0.82 | 0.88 | 0.82 |
| LD65 | 1.16 | 288.48 | 7.15 | 11.25 | 3.04 | 1.71 |
| LD66 | 0.99 | 16.81 | 1.82 | 7.74 | 1.16 | 0.84 |
| LD67 | 0.89 | 38.21 | 1.30 | 1.13 | 0.70 | 0.82 |
| LD68 | 0.80 | 147.41 | 1.66 | 4.45 | 1.12 | 2.27 |
| LD69 | 1.01 | 4210.68 | 11.37 | 7865.24 | 3.25 | 2.84 |
| LD70 | 1.38 | 2.89 | 2.44 | 2.11 | 1.55 | 1.96 |
| LD71 | 0.65 | 524.95 | 5.88 | 268.30 | 6.18 | 2.87 |
| LD72 | 0.92 | 796.81 | 9.85 | 89.55 | 1.13 | 2.05 |
| LD73 | 1.22 | 1.56 | 1.40 | 2.50 | 1.49 | 1.03 |
| LD74 | 0.91 | 140.64 | 2.83 | 42.70 | 0.97 | 2.01 |
| LD75 | 1.02 | 99.19 | 6.09 | 24.38 | 2.03 | 2.27 |
| LD76 | 0.49 | 59.93 | 1.72 | 24.74 | 0.72 | 4.66 |
| LD77 | 0.84 | 281.23 | 5.49 | 6.14 | 1.61 | 1.08 |
| LD78 | 0.77 | 257.48 | 6.63 | 109.68 | 0.40 | 1.37 |
| AD22 | 1.16 | 16.41 | 1.68 | 28.21 | 1.31 | 1.01 |
| AD52 | 1.23 | 464.44 | 3.09 | 282.13 | 6.41 | 1.40 |
| AD53 | 1.40 | 299.77 | 3.93 | 25.10 | 2.21 | 2.07 |
| AD54 | 0.99 | 255.78 | 1.93 | 55.96 | 0.80 | 1.19 |
| AD55 | 0.94 | 771.68 | 3.13 | 11.70 | 0.93 | 0.90 |
| AD56 | 1.61 | 137.71 | 3.52 | 71.58 | 11.72 | 2.05 |
| AD57 | 1.11 | 143.68 | 8.15 | 19.26 | 1.90 | 1.65 |
| AD58 | 1.46 | 363.91 | 2.71 | 6095.02 | 2.69 | 1.31 |
| AD59 | 1.00 | 32.18 | 5.15 | 12.47 | 1.75 | 1.63 |
| AD60 | 1.12 | 70.48 | 2.47 | 19.70 | 1.32 | 1.14 |
| AD61 | 1.18 | 2.87 | 1.17 | 4.75 | 1.09 | 0.80 |
| AD62 | 0.92 | 2.60 | 1.20 | 6.14 | 1.61 | 0.85 |
| AD63 | 1.33 | 29.75 | 4.04 | 5.66 | 1.50 | 2.29 |
| AD64 | 1.08 | 14.38 | 2.10 | 12.14 | 1.25 | 0.92 |
| AD66 | 1.08 | 146.55 | 2.95 | 872.38 | 2.92 | 1.90 |
| AD67 | 1.02 | 58.04 | 1.36 | 15.76 | 1.12 | 0.99 |
| AD68 | 1.23 | 309.39 | 2.19 | 25.25 | 3.19 | 1.23 |
| AD69 | 1.56 | 31.14 | 5.55 | 38.74 | 1.25 | 1.30 |
| AD70 | 0.98 | 2.23 | 1.06 | 3.42 | 1.26 | 0.85 |
| AD71 | 1.35 | 45.01 | 2.33 | 8.93 | 1.37 | 1.48 |
| AD72 | 1.08 | 795.11 | 2.79 | 83.99 | 3.08 | 1.06 |
| AD73 | 1.21 | 329.15 | 2.01 | 384.95 | 5.68 | 1.22 |
| AD74 | 1.10 | 140.84 | 1.61 | 17.39 | 0.82 | 1.61 |
| AD75 | 1.13 | 290.90 | 2.49 | 163.62 | 1.87 | 1.40 |
| AD76 | 1.10 | 1105.55 | 13.20 | 328.92 | 3.38 | 2.06 |
| AD77 | 0.99 | 1761.57 | 8.54 | 130.38 | 1.15 | 4.37 |
| AD78 | 0.90 | 15.33 | 1.08 | 19.12 | 0.68 | 0.96 |
| AD79 | 0.87 | 5.89 | 1.19 | 5.47 | 3.57 | 1.00 |
| AD80 | 1.27 | 280.85 | 3.28 | 22.23 | 2.35 | 0.99 |
| AD81 | 0.87 | 28.92 | 1.05 | 30.76 | 0.94 | 1.58 |

TABLE 5A
(validation samples; N = 63)

| | Confirmed | | | | | | |
|---|---|---|---|---|---|---|---|
| | Diagnosis | | | IGRA | | Biopsy/ | |
| | TB Active/ | | BCG | (QFN/ | | Microscopy | Culture |
| Patient ID | Latent | Diagnosis TB | Vaccinated | T-Spot) | PCR | Findings | Results |
| HD68 | healthy | not infected | unknown | negative | — | — | — |
| HD69 | healthy | not infected | no | negative | — | — | — |
| HD70 | healthy | not infected | unknown | negative | — | — | — |
| HD71 | healthy | not infected | yes | negative | — | — | — |
| HD72 | healthy | not infected | no | negative | — | — | — |
| HD73 | healthy | not infected | no | negative | — | — | — |
| HD74 | healthy | not infected | no | negative | — | — | — |
| HD75 | healthy | not infected | no | negative | — | — | — |
| HD76 | healthy | not infected | unknown | negative | — | — | — |
| HD77 | healthy | not infected | no | negative | — | — | — |
| HD78 | healthy | not infected | no | negative | — | — | — |
| HD79 | healthy | not infected | unknown | negative | — | — | — |
| HD80 | healthy | not infected | unknown | negative | — | — | — |
| HD81 | healthy | not infected | no | negative | — | — | — |
| HD82 | healthy | not infected | no | negative | — | — | — |
| HD83 | healthy | not infected | unknown | negative | — | — | — |
| HD84 | healthy | not infected | no | negative | — | — | — |
| HD85 | healthy | not infected | no | negative | — | — | — |
| HD86 | healthy | not infected | no | negative | — | — | — |
| HD87 | healthy | not infected | unknown | negative | — | — | — |
| HD88 | healthy | not infected | unknown | negative | — | — | — |
| HD89 | healthy | not infected | unknown | negative | — | — | — |
| HD90 | healthy | not infected | unknown | negative | — | — | — |
| LD79 | latent | treated as active | unknown | unknown | positve | | n.d. |
| | | TB previously: | | | | | |
| | | treatment 4 years | | | | | |
| | | ago | | | | | |
| LD81 | latent | — | — | — | — | — | — |
| LD82 | latent | — | yes | positive | n.d. | — | n.d. |
| LD83 | latent | — | no | positive | — | — | n.d. |
| LD84 | active | extrapulmonary | no | positive | positive | negative | negative |
| LD85 | latent | — | — | — | — | — | — |
| LD86 | latent | — | — | — | — | — | — |
| LD87 | latent | — | no | positive | n.d. | — | negative |
| LD88 | latent | — | unknown | positive | negative | — | negative |
| LD89 | latent | — | — | — | — | — | — |
| LD90 | latent | — | no | positive, — | | — | n.d. |
| LD91 | latent | — | yes | positive | n.d. | — | — |
| LD92 | latent | — | unknown | positive | negative | — | negative |
| LD93 | latent | — | unknown | positive | negative | — | n.d. |
| LD94 | latent | — | unknown | positive | negative | — | negative |
| LD95 | latent | — | yes | positive | — | — | negative |
| LD96 | latent | — | no | positive | — | — | — |
| LD97 | latent | — | unknown | positive | negative | — | negative |
| LD98 | latent | — | unknown | positive | n.d. | — | — |
| LD99 | latent | — | unknown | positive | negative | — | negative |
| AD66.2 | active | pulmonary | unknown | n.d. | positve | | positive |
| AD79.2 | active | pulmonary | unknown | n.d. | positve | n.d. | positive |
| AD82 | active | pulmonary | unknown | n.d. | positve | negative | negative |
| AD83 | active | extrapulmonary | unknown | negative | positve | positive | positve |
| AD84 | active | pulmonary | unknown | positve | positve | n.d. | positve |
| AD85 | active | pulmonary | unknown | positve | positve | — | positve |
| AD86 | active | — | unknown | positve | positve | negative | positve |
| AD87 | active | — | unknown | positve | positve | n.d. | positve |
| AD88 | active | pulmonary | unknown | positve | negative | negative | — |
| AD89 | — | — | no | negative | positve | n.d. | — |
| AD90 | active | pulmonary | no | negative | positve | — | positve |
| AD91 | active | — | — | positve | — | — | — |
| AD92 | active | — | — | positve | — | | — |
| AD93 | active | — | | positve | — | — | — |
| AD94 | active | pulmonary | unknown | positve | positve | | negative |
| AD95 | active | pulmonary, | | positve | positve | negative | positve |
| | | lymph nodes | | | | | |
| AD96 | active | — | — | positve | | — | — |
| AD97 | active | polmunary | no | positve | positve | — | — |
| AD98 | active | polmunary | no | positve | positve | — | positve |
| AD 100 | active | — | — | positve | — | — | — |

TABLE 5B
(validation samples; N = 63)

| | Fold | Fold | Fold | Fold | Fold | |
|---|---|---|---|---|---|---|
| Patient | change | change | change | change | change | Fold change |
| ID | (CTSS) | (CXCL10) | (GBP5) | (IFNG) | (IL19) | (TRIM69_nc) |
| HD68 | 0.78 | 0.70 | 0.73 | 0.63 | 1.11 | 1.30 |
| HD69 | 0.90 | 0.91 | 0.94 | 1.37 | 1.10 | 0.99 |
| HD70 | 0.89 | 1.01 | 0.88 | 0.75 | 0.71 | 0.82 |
| HD71 | 0.88 | 1.68 | 0.93 | 2.82 | 0.43 | 0.63 |
| HD72 | 0.83 | 0.88 | 0.80 | 0.51 | 1.01 | 0.79 |
| HD73 | 0.95 | 15.33 | 1.01 | 1.39 | 0.99 | 1.12 |
| HD74 | 0.93 | 1.14 | 0.97 | 0.97 | 0.87 | 0.92 |
| HD75 | 0.92 | 1.11 | 0.95 | 1.44 | 0.79 | 0.87 |
| HD76 | 1.10 | 1.80 | 1.05 | 0.92 | 1.44 | 1.11 |
| HD77 | 0.88 | 1.01 | 0.91 | 1.09 | 0.98 | 1.11 |
| HD78 | 1.07 | 1.00 | 0.91 | 0.87 | 0.64 | 1.63 |
| HD79 | 1.19 | 1.05 | 1.11 | 1.32 | 0.71 | 0.84 |
| HD80 | 0.93 | 1.37 | 0.88 | 1.11 | 0.50 | 0.97 |
| HD81 | 1.37 | 3.10 | 1.40 | 1.72 | 1.83 | 1.17 |
| HD82 | 1.03 | 5.23 | 0.98 | 1.30 | 1.07 | 0.97 |
| HD83 | 1.12 | 78.94 | 2.22 | 2.21 | 1.36 | 1.05 |
| HD84 | 0.98 | 0.43 | 0.94 | 0.69 | 0.77 | 1.31 |
| HD85 | 0.92 | 3.09 | 0.89 | 1.37 | 1.06 | 0.93 |
| HD86 | 0.91 | 0.87 | 0.83 | 0.97 | 1.03 | 0.96 |
| HD87 | 0.83 | 1.02 | 0.87 | 1.59 | 1.37 | 1.13 |
| HD88 | 1.07 | 0.91 | 1.03 | 0.80 | 1.10 | 0.98 |
| HD89 | 0.95 | 1.06 | 0.98 | 0.79 | 0.96 | 1.16 |
| HD90 | 0.81 | 3.29 | 0.77 | 0.38 | 1.71 | 1.20 |
| LD79 | 1.06 | 648.70 | 2.90 | 18.70 | 0.85 | 1.69 |
| LD81 | 0.92 | 132.93 | 1.76 | 12.24 | 0.68 | 1.18 |
| LD82 | 1.38 | 141.54 | 12.51 | 531.56 | 2.44 | 3.04 |
| LD83 | 1.26 | 74.58 | 5.24 | 8.85 | 0.93 | 1.70 |
| LD84 | 1.34 | 472.54 | 3.24 | 244.50 | 0.42 | 1.20 |
| LD85 | 0.80 | 1181.23 | 2.80 | 207.17 | 1.50 | 2.75 |
| LD86 | 1.07 | 155.76 | 2.39 | 27.05 | 1.42 | 0.98 |
| LD87 | 1.01 | 94.42 | 1.12 | 1.64 | 0.58 | 1.23 |
| LD88 | 1.07 | 3.04 | 1.61 | 4.17 | 0.71 | 1.35 |
| LD89 | 0.93 | 5.80 | 1.06 | 0.93 | 1.12 | 0.96 |
| LD90 | 1.38 | 166.22 | 8.64 | 81.19 | 1.53 | 2.09 |
| LD91 | 1.18 | 19.46 | 2.31 | 1.88 | 1.41 | 1.21 |
| LD92 | 1.03 | 1039.33 | 5.13 | 10.55 | 1.49 | 2.22 |
| LD93 | 1.55 | 1.56 | 1.61 | 14.08 | 1.71 | 1.01 |
| LD94 | 1.07 | 4.69 | 1.76 | 4.51 | 1.01 | 1.64 |
| LD95 | 1.08 | 1.38 | 1.06 | 1.23 | 0.86 | 1.02 |
| LD96 | 1.00 | 250.62 | 5.29 | 178.99 | 1.16 | 2.14 |
| LD97 | 0.96 | 1.07 | 1.04 | 1.12 | 0.29 | 1.21 |
| LD98 | 1.02 | 56.03 | 3.34 | 31.44 | 0.97 | 1.28 |
| LD99 | 1.09 | 83.93 | 7.26 | 15.16 | 6.08 | 2.17 |
| AD66.2 | 0.69 | 233.20 | 4.15 | 74.46 | 0.57 | 3.10 |
| AD79.2 | 1.04 | 258.20 | 3.96 | 14.49 | 0.73 | 1.01 |
| AD82 | 1.06 | 16.16 | 2.41 | 24.04 | 0.62 | 1.34 |
| AD83 | 1.05 | 3.79 | 1.04 | 3.56 | 1.08 | 0.79 |
| AD84 | 1.04 | 2.85 | 2.11 | 2.25 | 1.12 | 1.22 |
| AD85 | 2.32 | 1310.04 | 12.29 | 649.03 | 2.17 | 2.85 |
| AD86 | 1.06 | 199.74 | 1.85 | 79.85 | 2.91 | 1.09 |
| AD87 | 0.80 | 7.54 | 0.70 | 10.90 | 0.59 | 1.14 |
| AD88 | 1.27 | 767.67 | 2.26 | 143.48 | 0.65 | 1.36 |
| AD89 | 1.12 | 222.48 | 2.60 | 4.29 | 2.58 | 1.21 |
| AD90 | 1.05 | 116.48 | 1.69 | 147.51 | 2.12 | 0.93 |
| AD91 | 1.27 | 591.86 | 2.91 | 888.63 | 1.97 | 1.25 |
| AD92 | 1.04 | 193.90 | 2.85 | 11.69 | 2.00 | 1.61 |
| AD93 | 1.32 | 138.65 | 2.61 | 13.90 | 2.20 | 1.41 |
| AD94 | 0.85 | 4.81 | 1.75 | 6.34 | 1.00 | 1.23 |
| AD95 | 1.20 | 245.88 | 2.54 | 472.26 | 0.66 | 1.14 |
| AD96 | 1.19 | 92.50 | 4.05 | 1.88 | 3.38 | 1.29 |
| AD97 | 0.89 | 35.20 | 1.99 | 29.36 | 0.96 | 1.01 |
| AD98 | 1.20 | 4.26 | 1.22 | 2.12 | 0.98 | 1.18 |
| AD100 | 1.14 | 242.90 | 4.90 | 27.60 | 0.42 | 1.38 |

### Example 7: Infection Detection from PBMC Using Random-Forest Classifiyer

This example uses the same definitions and abbreviations as defined in Example 6.

The aim of this study was to establish classifiers for preselected marker combinations enabling a robust identification of individuals infected with tuberculosis pathogens. In this experiments freshly isolated peripheral blood mononuclear cells (PBMC) of 28 healthy (no previous contact with tuberculosis pathogens), 28 latently-infected and 30 actively-infected donors (training cohort) were stimulated with ESAT6 and CFP10 antigens as essentially described in example 1 (paragraph “stimulation of PBMCs). In this experiment, patients infected with pathogens causing tuberculosis were preselected with regard to substantial IFNG secretion from isolated PBMC upon stimulation with ESAT6/CFP10 proteins and thus patient collective was biased for the marker IFNG.

RNA isolation was performed as described in example 1. QPCR was performed as described in example 3. Random-forest classifiers were established using the software R [3.5.0] in combination with the packages ranger [0.9.0], readxl [1.1.0], stringr [1.3.0] and mlr [2.12.1]. The measurements of the samples described in Table 8 (training samples; N=86, including 28 healthy, 28 latently-infected and 30 actively-infected donors) were log 2-transformed. Afterwards, the function ranger( ) was used for training with the following parameters: number of trees=1e3, minimal node size=5, split rule=“extratrees” with the number of random splits set to 5 and the number of variables to possibly split at set to 1.

On these training samples, the random forest resulted in performances shown in Table 6. Considering a scoring based on the sum of sensitivity and specificity (last column), performances ranged from a score of 1.5367 for IL19 alone to a score of 1.8772 for IFNG/ncTRIM69. The performance of IFNG alone was very good (sensitivity: 97.87%; specificity: 89.15%; score sensitivity+specificity: 1.8702). The performance of IFNG alone was improved by the addition of either one additional marker (IFNG/ncTRIM69; sensitivity: 96.28%; specificity: 91.44%; score: 1.8772) or of four additional markers (CTSS/CXCL10/IFNG/IL19/ncTRIM69; sensitivity: 96.23%; specificity: 91.29%; score: 1.8752) (Table 6). Established classifiers were independently validated with RNA samples, obtained from specifically stimulated PBMC samples of 18 non infected healthy, 19 latently-infected and 19 actively-infected donors (Table 9); which have been generated as described before for the training cohort. The participants of this study were not preselected regarding levels of IFNG production and thus constitute a representative collective of tuberculosis patients. Herein, performances of preselected marker combinations (shown in Table 7) ranged from a score (sensitivity+specificity) of 1.813 for IFNG alone to 1.892 for IFNG/ncTRIM69. Unexpectedly, the performance of IFNG alone was independently improved by the combination with one additional marker, out of CXCL10, GBP5, CTSS or ncTRIM69, with the following performances: IFNG/ncTRIM69 (sensitivity: 94.7%; specificity: 94.4%; score sensitivity+specificity: 1.892), CXCL10/IFNG (sensitivity: 92.1%; specificity: 94.4%; score sensitivity+specificity: 1.865), GBP5/IFNG (sensitivity: 89.5%; specificity: 94.4%; score sensitivity+specificity: 1.839), and CTSS/IFNG (sensitivity: 89.5%; specificity: 94.4%; score sensitivity+specificity: 1.839). In addition, multiple combinations of IFNG with 2 to 4 additional markers (out of CXCL10, GBP5, CTSS, ncTRIM69, IL19) showed performances superior to that of IFNG alone (Table 7).

Thus, established classifiers for described marker combinations allow a robust identification of patients infected by tuberculosis pathogens applying PBMC samples.

TABLE 6
PBMC-based classifier training set (28 non-infected/28 latent TB/30 active TB; N = 86)

| | | | | | Scoring: |
|---|---|---|---|---|---|
| | | infected.recall | non.infected.recall | | sum |
| genes | accuracy | (sensitivity) | (specificity) | AUC | sens + spec |
| IFNG/ncTRIM69 | 0.9470 | 0.9628 | 0.9144 | 0.9672 | 1.8772 |
| CTSS/CXCL10/IFNG/IL19/ncTRIM69 | 0.9460 | 0.9623 | 0.9129 | 0.9789 | 1.8752 |
| IFNG | 0.9505 | 0.9787 | 0.8915 | 0.9837 | 1.8702 |
| CXCL10/IFNG/IL19/ncTRIM69 | 0.9441 | 0.9638 | 0.9037 | 0.9791 | 1.8676 |
| IFNG/IL19 | 0.9431 | 0.9610 | 0.9061 | 0.9793 | 1.8671 |
| CTSS/CXCL10/IFNG/IL19 | 0.9437 | 0.9628 | 0.9029 | 0.9839 | 1.8657 |
| CTSS/IFNG | 0.9390 | 0.9526 | 0.9124 | 0.9746 | 1.8650 |
| CXCL10/IFNG/IL19 | 0.9413 | 0.9639 | 0.8931 | 0.9831 | 1.8570 |
| CTSS/CXCL10/GBP5/IFNG/IL19 | 0.9398 | 0.9618 | 0.8944 | 0.9836 | 1.8562 |
| IFNG/IL19/ncTRIM69 | 0.9371 | 0.9571 | 0.8968 | 0.9755 | 1.8539 |
| GBP5/IFNG/IL19/ncTRIM69 | 0.9328 | 0.9445 | 0.9089 | 0.9792 | 1.8535 |
| CTSS/GPB5/IFNG/IL19/ncTRIM69 | 0.9320 | 0.9435 | 0.9087 | 0.9774 | 1.8521 |
| GPB5/IFNG/IL19 | 0.9362 | 0.9543 | 0.8976 | 0.9808 | 1.8519 |
| CTSS/IFNG/IL19 | 0.9382 | 0.9611 | 0.8908 | 0.9785 | 1.8519 |
| CXCL10/GBP5/IFNG/IL19/ncTRIM69 | 0.9384 | 0.9605 | 0.8913 | 0.9798 | 1.8518 |
| GPB5/IFNG | 0.9373 | 0.9592 | 0.8913 | 0.9832 | 1.8505 |
| CXCL10/GPB5/IFNG/IL19 | 0.9361 | 0.9560 | 0.8944 | 0.9830 | 1.8504 |
| CTSS/CXCL10/IL19 | 0.9360 | 0.9577 | 0.8916 | 0.9811 | 1.8493 |
| CXCL10/IFNG/ncTRIM69 | 0.9367 | 0.9587 | 0.8905 | 0.9761 | 1.8493 |
| CXCL10/IFNG | 0.9363 | 0.9617 | 0.8841 | 0.9802 | 1.8458 |
| CTSS/CXCL10/GBP5/IFNG/IL19/ | 0.9333 | 0.9543 | 0.8896 | 0.9810 | 1.8439 |
| ncTRIM69 | | | | | |
| CXCL10/IL19 | 0.9351 | 0.9602 | 0.8837 | 0.9806 | 1.8439 |
| CTSS/GPB5/IFNG/IL19 | 0.9323 | 0.9506 | 0.8933 | 0.9808 | 1.8439 |
| CTSS/GBP5/IFNG | 0.9319 | 0.9518 | 0.8896 | 0.9790 | 1.8414 |
| GPB5/IFNG/ncTRIM69 | 0.9299 | 0.9485 | 0.8911 | 0.9787 | 1.8396 |
| CXCL10/GBP5/IFNG/ncTRIM69 | 0.9298 | 0.9496 | 0.8889 | 0.9779 | 1.8385 |
| CTSS/CXCL10/IFNG | 0.9311 | 0.9524 | 0.8853 | 0.9807 | 1.8378 |
| CTSS/CXCL10/IFNG/ncTRIM69 | 0.9280 | 0.9458 | 0.8907 | 0.9789 | 1.8365 |
| CXCL10/GBP5/IFNG | 0.9285 | 0.9487 | 0.8864 | 0.9817 | 1.8351 |
| CXCL10/IL19/ncTRIM69 | 0.9307 | 0.9589 | 0.8736 | 0.9783 | 1.8325 |
| CTSS/GBP5/IFNG/ncTRIM69 | 0.9254 | 0.9437 | 0.8871 | 0.9759 | 1.8308 |
| CTSS/CXCL10/IL19/ncTRIM69 | 0.9267 | 0.9496 | 0.8811 | 0.9763 | 1.8307 |
| CTSS/CXCL10/GBP5/IFNG | 0.9258 | 0.9474 | 0.8807 | 0.9798 | 1.8280 |
| CTSS/IFNG/ncTRIM69 | 0.9201 | 0.9357 | 0.8901 | 0.9674 | 1.8259 |
| CXCL10/GPB5/IL19 | 0.9253 | 0.9496 | 0.8761 | 0.9812 | 1.8258 |
| CTSS/IFNG/IL19/ncTRIM69 | 0.9233 | 0.9458 | 0.8781 | 0.9723 | 1.8240 |
| CTSS/CXCL10/GBP5/IFNG/ncTRIM69 | 0.9204 | 0.9387 | 0.8819 | 0.9797 | 1.8206 |
| GBP5/IL19/ncTRIM69 | 0.9151 | 0.9312 | 0.8841 | 0.9720 | 1.8153 |
| CTSS/CXCL10/GBP5/IL19 | 0.9210 | 0.9482 | 0.8640 | 0.9816 | 1.8122 |
| GBP5/IL19 | 0.9130 | 0.9335 | 0.8716 | 0.9743 | 1.8051 |
| CTSS/GPB5/IL19/ncTRIM69 | 0.9113 | 0.9310 | 0.8735 | 0.9707 | 1.8045 |
| CXCL10/GPB5/IL19/ncTRIM69 | 0.9189 | 0.9508 | 0.8529 | 0.9794 | 1.8037 |
| CTSS/GPB5/IL19 | 0.9099 | 0.9371 | 0.8544 | 0.9750 | 1.7915 |
| CTSS/CXCL10/GBP5/IL19/ncTRIM69 | 0.9086 | 0.9420 | 0.8405 | 0.9779 | 1.7825 |
| CTSS/CXCL10/GBP5 | 0.8898 | 0.9236 | 0.8209 | 0.9752 | 1.7445 |
| CTSS/GPB5 | 0.8871 | 0.9175 | 0.8239 | 0.9697 | 1.7414 |
| CXCL10/GPB5/ncTRIM69 | 0.8875 | 0.9265 | 0.8084 | 0.9714 | 1.7349 |
| CTSS/CXCL10 | 0.8837 | 0.9152 | 0.8188 | 0.9723 | 1.7340 |
| CXCL10/GBP5 | 0.8884 | 0.9296 | 0.8035 | 0.9724 | 1.7330 |
| GBP5 | 0.8848 | 0.9212 | 0.8104 | 0.9723 | 1.7316 |
| CTSS/GPB5/ncTRIM69 | 0.8792 | 0.9105 | 0.8156 | 0.9633 | 1.7261 |
| CTSS/CXCL10/ncTRIM69 | 0.8794 | 0.9150 | 0.8095 | 0.9687 | 1.7244 |
| GPB5/ncTRIM69 | 0.8794 | 0.9148 | 0.8064 | 0.9630 | 1.7212 |
| CTSS/CXCL10/GPB5/ncTRIM69 | 0.8806 | 0.9196 | 0.8011 | 0.9743 | 1.7207 |
| CXCL10/ncTRIM69 | 0.8788 | 0.9170 | 0.8017 | 0.9625 | 1.7187 |
| CXCL10 | 0.8673 | 0.8995 | 0.7997 | 0.9682 | 1.6992 |
| CTSS/IL19/ncTRIM69 | 0.8583 | 0.8997 | 0.7753 | 0.9371 | 1.6750 |
| CTSS/ncTRIM69 | 0.8424 | 0.8649 | 0.7969 | 0.9157 | 1.6618 |
| IL19/ncTRIM69 | 0.8520 | 0.9047 | 0.7437 | 0.9340 | 1.6484 |
| TRIM69 | 0.8348 | 0.8670 | 0.7691 | 0.8767 | 1.6361 |
| CTSS/IL19 | 0.8359 | 0.8994 | 0.7039 | 0.9306 | 1.6033 |
| CTSS | 0.8136 | 0.8602 | 0.7203 | 0.8987 | 1.5805 |
| IL19 | 0.8028 | 0.8659 | 0.6708 | 0.8911 | 1.5367 |

TABLE 7
PBMC-based classifier test set (18 non-infected/19 latent TB/19 active TB; N = 56)

| | | | | | scoring: |
|---|---|---|---|---|---|
| | | infected.recall | noninfected.recall | | sum |
| Genes | Accuracy | (sensitivity) | (specificity) | AUC | sens + spec |
| IFNG/ncTRIM69 | 0.946 | 0.947 | 0.944 | 0.963 | 1.892 |
| CXCL10/IFNG/ncTRIM69 | 0.946 | 0.947 | 0.944 | 0.961 | 1.892 |
| CXCL10/IFNG | 0.929 | 0.921 | 0.944 | 0.976 | 1.865 |
| CTSS/CXCL10/IFNG | 0.929 | 0.921 | 0.944 | 0.965 | 1.865 |
| CTSS/CXCL10/IFNG/IL19/ncTRIM69 | 0.929 | 0.921 | 0.944 | 0.962 | 1.865 |
| CTSS/IFNG/ncTRIM69 | 0.929 | 0.921 | 0.944 | 0.953 | 1.865 |
| CTSS/CXCL10/IFNG/ncTRIM69 | 0.929 | 0.921 | 0.944 | 0.953 | 1.865 |
| CTSS/CXCL10/GBP5/IFNG/ncTRIM69 | 0.929 | 0.921 | 0.944 | 0.950 | 1.865 |
| GBP5/IFNG | 0.911 | 0.895 | 0.944 | 0.974 | 1.839 |
| CXCL10/IFNG/IL19 | 0.911 | 0.895 | 0.944 | 0.974 | 1.839 |
| CXCL10/IFNG/IL19/ncTRIM69 | 0.911 | 0.895 | 0.944 | 0.972 | 1.839 |
| CTSS/GBP5/IFNG | 0.911 | 0.895 | 0.944 | 0.965 | 1.839 |
| CXCL10/GBP5/IFNG | 0.911 | 0.895 | 0.944 | 0.964 | 1.839 |
| CTSS/IFNG | 0.911 | 0.895 | 0.944 | 0.963 | 1.839 |
| CTSS/CXCL10/GBP5/IFNG | 0.911 | 0.895 | 0.944 | 0.962 | 1.839 |
| GBP5/IFNG/ncTRIM69 | 0.911 | 0.895 | 0.944 | 0.955 | 1.839 |
| CXCL10/GBP5/IFNG/ncTRIM69 | 0.911 | 0.895 | 0.944 | 0.955 | 1.839 |
| IFNG | 0.893 | 0.868 | 0.944 | 0.969 | 1.813 |

TABLE 8
(training samples; N = 86)

| | | Fold | Fold | Fold | Fold | Fold | |
|---|---|---|---|---|---|---|---|
| Patient | Diagnosis | change | change | change | change | change | Fold change |
| ID | TB | (CTSS) | (CXCL10) | (GBP5) | (IFNG) | (IL19) | (ncTRIM69) |
| HD1 | healthy | 1.10 | 1.46 | 1.10 | 1.00 | 1.04 | 1.02 |
| HD2 | healthy | 1.19 | 1.29 | 1.20 | 1.17 | 1.40 | 1.31 |
| HD3 | healthy | 1.07 | 1.95 | 1.03 | 1.43 | 1.37 | 1.04 |
| HD4 | healthy | 1.01 | 0.88 | 0.88 | 1.12 | 0.85 | 0.89 |
| HD5 | healthy | 0.90 | 1.46 | 0.93 | 1.33 | 1.07 | 1.15 |
| HD6 | healthy | 1.02 | 0.70 | 0.93 | 1.12 | 0.89 | 1.19 |
| HD7 | healthy | 0.97 | 0.77 | 1.00 | 0.98 | 0.91 | 0.94 |
| HD8 | healthy | 1.52 | 2.88 | 1.99 | 1.28 | 1.79 | 1.81 |
| HD9 | healthy | 1.06 | 1.59 | 1.06 | 1.33 | 1.06 | 1.12 |
| HD10 | healthy | 0.98 | 1.93 | 1.04 | 0.93 | 1.06 | 0.85 |
| HD11 | healthy | 1.04 | 1.82 | 1.33 | 1.97 | 0.99 | 0.90 |
| HD13 | healthy | 1.20 | 1.42 | 1.19 | 1.35 | 1.56 | 1.31 |
| HD14 | healthy | 1.52 | 1.48 | 1.51 | 1.50 | 1.85 | 1.42 |
| HD15 | healthy | 0.96 | 18.18 | 2.82 | 2.42 | 0.80 | 0.91 |
| HD16 | healthy | 0.95 | 0.61 | 1.17 | 0.95 | 0.88 | 1.19 |
| HD17 | healthy | 0.96 | 2.20 | 1.06 | 1.12 | 0.75 | 1.11 |
| HD18 | healthy | 0.99 | 1.12 | 1.00 | 1.12 | 0.72 | 1.32 |
| HD19 | healthy | 1.13 | 0.90 | 1.23 | 1.02 | 1.08 | 1.43 |
| HD20 | healthy | 1.03 | 7.04 | 1.70 | 1.77 | 1.06 | 0.97 |
| HD21 | healthy | 1.08 | 1.17 | 1.04 | 1.23 | 1.01 | 1.17 |
| HD22 | healthy | 1.22 | 4.21 | 2.34 | 1.46 | 1.24 | 1.27 |
| HD23 | healthy | 0.92 | 1.72 | 1.26 | 2.16 | 1.04 | 1.09 |
| HD24 | healthy | 1.28 | 12.39 | 4.03 | 6.39 | 1.56 | 2.57 |
| HD25 | healthy | 0.89 | 15.62 | 1.94 | 7.27 | 1.27 | 1.37 |
| HD26 | healthy | 1.06 | 1.02 | 1.04 | 1.35 | 2.56 | 1.14 |
| HD27 | healthy | 0.97 | 1.21 | 0.97 | 0.87 | 0.95 | 0.98 |
| HD29 | healthy | 0.91 | 0.85 | 0.90 | 0.95 | 0.80 | 0.87 |
| HD30 | healthy | 0.94 | 0.94 | 0.97 | 1.13 | 0.89 | 0.88 |
| LD1 | latent | 1.59 | 34.02 | 10.46 | 11.73 | 2.15 | 2.34 |
| LD2 | latent | 2.49 | 277.08 | 42.90 | 295.29 | 8.00 | 2.95 |
| LD3 | latent | 1.76 | 353.96 | 17.60 | 53.40 | 1.52 | 2.11 |
| LD4 | latent | 1.78 | 336.46 | 20.29 | 26.12 | 2.00 | 2.04 |
| LD5 | latent | 1.69 | 113.78 | 8.02 | 15.28 | 3.83 | 1.86 |
| LD6 | latent | 1.06 | 9.28 | 2.61 | 3.33 | 2.20 | 1.38 |
| LD7 | latent | 1.56 | 130.28 | 12.77 | 51.56 | 15.24 | 1.83 |
| LD8 | latent | 1.16 | 2.62 | 1.90 | 10.30 | 5.84 | 1.33 |
| LD10 | healthy | 1.43 | 69.29 | 6.99 | 7.70 | 3.71 | 2.09 |
| LD11 | latent | 2.92 | 133.46 | 35.80 | 47.42 | 17.30 | 2.77 |
| LD12 | latent | 0.87 | 7.41 | 2.82 | 6.92 | 3.41 | 0.93 |
| LD13 | latent | 1.89 | 51.09 | 13.35 | 22.87 | 7.01 | 2.01 |
| LD14 | latent | 4.77 | 287.61 | 78.65 | 189.53 | 24.64 | 4.77 |
| LD15 | latent | 3.11 | 261.25 | 25.31 | 77.21 | 12.05 | 2.53 |
| LD16 | latent | 2.04 | 14.56 | 8.26 | 6.13 | 4.76 | 1.95 |
| LD17 | latent | 1.44 | 222.09 | 9.83 | 22.37 | 4.14 | 1.99 |
| LD18 | latent | 2.26 | 1799.98 | 64.22 | 99.29 | 2.98 | 5.92 |
| LD19 | latent | 1.35 | 504.56 | 12.14 | 62.26 | 2.05 | 2.05 |
| LD20 | latent | 1.13 | 84.17 | 5.98 | 17.36 | 0.78 | 1.42 |
| LD22 | latent | 1.09 | 27.40 | 11.98 | 29.86 | 3.64 | 1.96 |
| LD23 | latent | 1.49 | 161.41 | 10.57 | 35.97 | 1.69 | 1.60 |
| LD24 | latent | 1.27 | 78.84 | 5.40 | 3.47 | 1.56 | 2.24 |
| LD25 | latent | 1.18 | 31.47 | 7.33 | 7.26 | 1.15 | 1.86 |
| LD26 | latent | 1.62 | 808.91 | 9.39 | 25.25 | 0.96 | 2.71 |
| LD27 | latent | 1.70 | 76.82 | 8.77 | 8.25 | 1.23 | 1.60 |
| LD28 | latent | 1.02 | 27.50 | 1.65 | 2.83 | 1.47 | 1.23 |
| LD29 | latent | 1.31 | 26.96 | 3.81 | 7.32 | 1.36 | 1.94 |
| LD30 | latent | 1.25 | 15.53 | 3.75 | 5.85 | 1.58 | 1.25 |
| AD1 | active | 1.83 | 226.20 | 26.08 | 61.75 | 11.77 | 2.48 |
| AD2 | active | 1.85 | 747.33 | 46.90 | 93.41 | 3.02 | 5.39 |
| AD3 | active | 1.59 | 131.95 | 14.28 | 78.18 | 5.20 | 1.88 |
| AD4 | active | 2.26 | 207.71 | 23.66 | 192.11 | 7.69 | 2.17 |
| AD5 | active | 1.70 | 120.23 | 23.75 | 274.38 | 7.84 | 3.07 |
| AD6 | active | 1.61 | 332.49 | 13.45 | 45.42 | 2.47 | 2.09 |
| AD7 | active | 2.04 | 49.34 | 16.30 | 89.47 | 1.73 | 1.28 |
| AD8 | active | 2.82 | 142.61 | 11.15 | 253.60 | 3.66 | 2.75 |
| AD9 | active | 3.13 | 163.23 | 33.73 | 47.14 | 4.38 | 3.53 |
| AD10 | active | 2.36 | 30.43 | 12.42 | 121.93 | 8.13 | 1.41 |
| AD11 | active | 1.46 | 37.34 | 6.41 | 15.59 | 2.06 | 2.63 |
| AD12 | active | 1.15 | 4.38 | 2.76 | 2.65 | 0.71 | 1.40 |
| AD13 | active | 1.17 | 158.37 | 14.37 | 22.17 | 1.09 | 2.86 |
| AD14 | active | 1.98 | 174.28 | 20.86 | 72.42 | 3.68 | 2.01 |
| AD15 | active | 1.89 | 39.43 | 11.55 | 102.78 | 2.75 | 1.90 |
| AD16 | active | 2.02 | 167.96 | 16.43 | 26.77 | 2.11 | 3.34 |
| AD17 | active | 1.31 | 63.37 | 6.74 | 4.08 | 2.28 | 2.61 |
| AD18 | active | 0.83 | 1.93 | 1.30 | 1.65 | 1.35 | 0.83 |
| AD19 | active | 2.47 | 28.15 | 7.71 | 35.49 | 3.41 | 2.38 |
| AD20 | active | 1.23 | 18.73 | 3.76 | 12.18 | 1.63 | 1.38 |
| AD21 | active | 2.25 | 289.81 | 22.34 | 423.25 | 16.20 | 2.89 |
| AD22 | active | 2.60 | 149.74 | 21.75 | 152.36 | 3.91 | 1.88 |
| AD23 | active | 2.14 | 99.36 | 27.85 | 34.93 | 6.64 | 2.65 |
| AD24 | active | 2.22 | 26.25 | 17.12 | 45.96 | 1.76 | 2.68 |
| AD25 | active | 1.80 | 332.21 | 10.62 | 146.07 | 3.59 | 2.17 |
| AD26 | active | 1.32 | 52.56 | 5.41 | 15.86 | 1.76 | 2.03 |
| AD27 | active | 2.83 | 247.86 | 38.60 | 859.69 | 3.08 | 2.53 |
| AD28 | active | 2.39 | 265.97 | 27.91 | 101.67 | 4.35 | 1.93 |
| AD29 | active | 1.04 | 14.19 | 1.78 | 3.98 | 1.50 | 1.06 |
| AD30 | active | 1.96 | 646.46 | 26.92 | 51.74 | 3.10 | 2.78 |

TABLE 9
(validation samples; N = 56)

| | | Fold | Fold | Fold | Fold | Fold | |
|---|---|---|---|---|---|---|---|
| Patient | Diagnosis | change | change | change | change | change | Fold change |
| ID | TB | (CTSS) | (CXCL10) | (GBP5) | (IFNG) | (IL19) | (ncTRIM69) |
| HD31 | healthy | 0.95 | 0.91 | 0.94 | 1.11 | 1.07 | 0.86 |
| HD33 | healthy | 0.95 | 1.21 | 0.92 | 1.12 | 0.73 | 0.90 |
| HD34 | healthy | 0.93 | 1.48 | 1.11 | 1.50 | 0.88 | 1.07 |
| HD35 | healthy | 1.04 | 2.66 | 1.24 | 2.11 | 0.95 | 0.92 |
| HD36 | healthy | 1.23 | 7.82 | 1.56 | 1.79 | 1.38 | 1.46 |
| HD37 | healthy | 1.07 | 0.73 | 0.96 | 0.93 | 0.95 | 1.01 |
| HD38 | healthy | 0.67 | 0.85 | 0.77 | 1.29 | 0.72 | 0.88 |
| HD39 | healthy | 1.09 | 9.77 | 4.20 | 6.79 | 1.02 | 1.41 |
| HD40 | healthy | 0.98 | 0.60 | 0.94 | 0.67 | 0.82 | 1.07 |
| HD41 | healthy | 1.03 | 2.19 | 1.11 | 2.09 | 1.59 | 1.03 |
| HD42 | healthy | 1.06 | 1.22 | 1.07 | 0.89 | 0.97 | 1.05 |
| HD43 | healthy | 0.93 | 0.94 | 0.99 | 0.88 | 1.38 | 0.77 |
| HD44 | healthy | 1.17 | 2.28 | 1.44 | 0.96 | 1.50 | 1.70 |
| HD45 | healthy | 1.17 | 1.36 | 1.31 | 1.25 | 1.85 | 1.15 |
| HD46 | healthy | 0.81 | 0.93 | 0.90 | 0.90 | 1.07 | 0.87 |
| HD47 | healthy | 1.08 | 1.31 | 0.97 | 0.80 | 1.57 | 0.61 |
| HD49 | healthy | 0.97 | 0.94 | 0.95 | 0.98 | 0.58 | 1.05 |
| HD50 | healthy | 0.96 | 0.67 | 0.90 | 0.83 | 0.92 | 1.07 |
| LD31 | latent | 3.01 | 594.75 | 55.53 | 40.50 | 8.73 | 6.33 |
| LD32 | latent | 1.19 | 75.56 | 5.07 | 4.94 | 5.78 | 1.55 |
| LD33 | latent | 1.29 | 5.25 | 2.90 | 25.76 | 5.17 | 1.43 |
| LD34 | latent | 1.60 | 128.28 | 28.31 | 49.46 | 2.89 | 3.32 |
| LD35 | latent | 1.33 | 13.45 | 5.40 | 8.63 | 1.74 | 2.00 |
| LD36 | latent | 1.92 | 239.05 | 30.42 | 33.99 | 15.56 | 2.76 |
| LD37 | latent | 1.27 | 32.99 | 6.92 | 5.19 | 2.58 | 2.63 |
| LD38 | latent | 1.06 | 9.73 | 1.70 | 4.24 | 1.19 | 1.11 |
| LD39 | latent | 1.30 | 382.71 | 41.69 | 40.02 | 3.08 | 2.59 |
| LD40 | latent | 1.70 | 274.72 | 25.14 | 1.69 | 1.61 | 2.81 |
| LD41 | latent | 1.13 | 5.13 | 2.59 | 2.99 | 2.07 | 1.80 |
| LD42 | latent | 1.63 | 236.12 | 15.71 | 32.28 | 3.11 | 2.45 |
| LD43 | latent | 3.18 | 219.59 | 32.65 | 547.77 | 46.94 | 2.39 |
| LD44 | latent | 1.03 | 0.66 | 0.84 | 1.27 | 1.19 | 0.93 |
| LD45 | latent | 1.15 | 8.01 | 1.65 | 2.47 | 1.05 | 1.42 |
| LD46 | latent | 2.10 | 162.57 | 32.63 | 74.10 | 3.38 | 2.01 |
| LD47 | latent | 1.38 | 94.41 | 7.78 | 25.45 | 1.42 | 1.07 |
| LD48 | latent | 1.04 | 5.93 | 2.73 | 3.43 | 0.93 | 1.35 |
| LD49 | latent | 1.68 | 284.55 | 15.09 | 13.84 | 1.46 | 2.97 |
| AD31 | active | 1.29 | 13.79 | 5.90 | 11.14 | 1.47 | 1.69 |
| AD32 | active | 1.98 | 246.15 | 11.16 | 93.55 | 1.68 | 1.95 |
| AD33 | active | 1.88 | 191.78 | 11.34 | 23.04 | 2.44 | 2.03 |
| AD34 | active | 3.18 | 368.43 | 14.75 | 64.56 | 2.25 | 3.86 |
| AD35 | active | 1.97 | 51.22 | 5.06 | 30.11 | 3.46 | 2.81 |
| AD36 | active | 1.15 | 8.57 | 2.69 | 7.20 | 1.28 | 1.14 |
| AD37 | active | 2.17 | 465.26 | 19.66 | 114.49 | 3.82 | 2.93 |
| AD38 | active | 2.14 | 247.85 | 9.22 | 23.57 | 2.24 | 2.42 |
| AD39 | active | 0.75 | 17.15 | 1.35 | 3.66 | 0.93 | 1.36 |
| AD40 | active | 1.26 | 30.34 | 3.54 | 12.53 | 1.13 | 1.68 |
| AD41 | active | 1.00 | 1.33 | 1.15 | 1.45 | 1.29 | 1.16 |
| AD42 | active | 1.81 | 714.18 | 14.17 | 251.23 | 5.47 | 2.38 |
| AD43 | active | 1.46 | 3.22 | 1.77 | 43.65 | 26.29 | 1.22 |
| AD44 | active | 2.77 | 938.76 | 56.04 | 75.31 | 3.28 | 3.77 |
| AD45 | active | 0.90 | 5.74 | 1.29 | 2.42 | 0.84 | 1.40 |
| AD46 | active | 0.53 | 46.20 | 3.33 | 10.10 | 0.58 | 0.98 |
| AD47 | active | 1.37 | 301.74 | 22.13 | 31.37 | 1.16 | 1.96 |
| AD49 | active | 2.05 | 644.24 | 37.56 | 139.56 | 10.78 | 2.12 |
| AD50 | active | 2.94 | 162.88 | 17.14 | 495.31 | 2.64 | 2.71 |

### Example 8: Infection Detection from Whole Blood Using ncTRIM69-Composing Random-Forest Classifiyer

This example uses the same definitions and abbreviations as defined in Example 6.

The aim of this study was to establish classifiers for preselected ncTRIM69 composing marker combinations enabling a robust identification of individuals infected with tuberculosis pathogens.

In this experiments anticoagulated whole blood samples of 27 healthy donors without known contact with tuberculosis pathogens as well as 30 latently-infected and 30 actively-infected donors (training cohort) were stimulated with ESAT6 and CFP10 antigens as essentially described in example 1 (paragraph “stimulation of PBMCs). In this experiment, patients infected with pathogens causing tuberculosis were preselected with regard to substantial IFNG secretion from isolated PBMC upon stimulation with ESAT6/CFP10 proteins and thus patient collective was biased for the marker IFNG.

RNA isolation was performed as described in example 1. QPCR was performed as described in example 3. Then, random-forest classifiers were established using the software R [3.5.0] in combination with the packages ranger [0.9.0], readxl [1.1.0], stringr [1.3.0] and mlr [2.12.1]. The measurements of the samples described in Table 4/7B (training samples; N=87, including 27 healthy, 30 latently-infected and 30 actively-infected donors) were log 2-transformed. Afterwards, the function ranger( ) was used for training with the following parameters: number of trees=1e3, minimal node size=5, split rule=“extratrees” with the number of random splits set to 5 and the number of variables to possibly split at set to 1. The performance of the Random Forest classifier generated on these training samples, for ncTRIM69 alone or in combination with other genes, out of CXCL10, GBP5, IFNG, CTSS and IL19, is shown in Table 10.

Established classifiers were independently validated with RNA samples, obtained from specifically stimulated anticoagulated whole blood of 23 healthy, 20 latently-infected and 20 actively-infected donors (Table 5A/B); which have been generated as described before for the training cohort. ncTRIM69 alone had a discriminating power for infection recognition with a sensitivity of 72.50%, a specificity of 65.22% and a score (sensitivity+specificity) of 1.3772 (Table 11). The addition of ncTRIM69 to at least 8 combinations of genes, comprising any of the following markers: CXCL10, GBP5, IFNG, CTSS and IL19, improved their performance in terms of sensitivity and/or specificity. For instance, the performance of GBP5/IFNG (sensitivity: 87.50%; specificity: 91.30%; score sensitivity+specificity: 1.7880) was improved by the addition of ncTRIM69 (sensitivity: 87.50%; specificity: 95.65%; score sensitivity+specificity: 1.8315). Also, the performance of CXCL10/GBP5/IFNG (sensitivity: 90.00%; specificity: 91.30%; score sensitivity+specificity: 1.8130) was improved by the combination with ncTRIM69 (sensitivity: 90.00%; specificity: 95.65%; score sensitivity+specificity: 1.8565). Similarly, the performance of CTSS/CXCL10/GBP5/IFNG/IL19, of CTSS/GBP5/IFNG/IL19, of CTSS/GBP5/IFNG, of CTSS/CXCL10/GBP5, of CXCL10/GBP5/IFNG/IL19, and of CTSS/CXCL10/GBP5/IFNG was improved by the addition of ncTRIM69 (Table 11).

Thus, established classifiers for described ncTRIM69 composing marker combinations allow a robust identification of patients infected by tuberculosis pathogens applying whole blood samples.

TABLE 10
Blood-based classifier training set (27 non-infected/30 latent TB/30 active TB; N = 87)

| | | | | | Scoring: |
|---|---|---|---|---|---|
| | | infected.recall | noninfected.recall | | sum |
| Genes | Accuracy | (sensitivity) | (specificity) | AUC | sens+spec |
| CXCL10/GPB5/IFNG | 0.9283 | 0.9227 | 0.9409 | 0.9709 | 1.8636 |
| CXCL10/GPB5/IFNG/ncTRIM69 | 0.9226 | 0.9213 | 0.9253 | 0.9739 | 1.8467 |
| CXCL10/GPB5/IFNG/IL19/ncTRIM69 | 0.9197 | 0.9203 | 0.9193 | 0.9679 | 1.8397 |
| CTSS/CXCL10/GPB5/IFNG | 0.9197 | 0.9233 | 0.9125 | 0.9650 | 1.8359 |
| CXCL10/GBP5/IFNG/IL19 | 0.9141 | 0.9190 | 0.9036 | 0.9669 | 1.8226 |
| CTSS/CXCL10/GPB5/IFNG/ncTRIM69 | 0.9132 | 0.9193 | 0.8999 | 0.9681 | 1.8192 |
| CTSS/CXCL10/IFNG/IL19/ncTRIM69 | 0.9009 | 0.9167 | 0.8685 | 0.9607 | 1.7852 |
| CTSS/CXCL10/IFNG/IL19 | 0.8946 | 0.9030 | 0.8791 | 0.9573 | 1.7821 |
| CTSS/CXCL10/GBP5/IFNG/IL19 | 0.8967 | 0.9107 | 0.8680 | 0.9612 | 1.7787 |
| CTSS/CXCL10/GPB5/IFNG/IL19/ | 0.8935 | 0.9140 | 0.8497 | 0.9641 | 1.7637 |
| ncTRIM69 | | | | | |
| CTSS/CXCL10/GBP5/ncTRIM69 | 0.8858 | 0.8993 | 0.8571 | 0.9575 | 1.7564 |
| GBP5/IFNG | 0.8823 | 0.8960 | 0.8524 | 0.9594 | 1.7484 |
| IFNG/ncTRIM69 | 0.8813 | 0.8947 | 0.8535 | 0.9485 | 1.7481 |
| CTSS/CXCL10/GPB5 | 0.8810 | 0.8987 | 0.8432 | 0.9419 | 1.7419 |
| GBP5/IFNG/ncTRIM69 | 0.8810 | 0.8990 | 0.8427 | 0.9627 | 1.7417 |
| CTSS/GBP5/IFNG | 0.8801 | 0.9020 | 0.8364 | 0.9541 | 1.7384 |
| IFNG | 0.8753 | 0.8873 | 0.8499 | 0.9312 | 1.7372 |
| ncTRIM69 | 0.6340 | 0.7607 | 0.3528 | 0.6990 | 1.1135 |

TABLE 11
Blood-based classifier test set (23 non-infected/20 latent TB/20 active TB; N = 63)

| | | | | | scoring: |
|---|---|---|---|---|---|
| | | infected.recall | noninfected.recall | | sum |
| Genes | Accuracy | (sensitivity) | (specificity) | AUC | sens + spec |
| CXCL10/GPB5/IFNG/ncTRIM69 | 0.9206 | 0.9000 | 0.9565 | 0.9489 | 1.8565 |
| CTSS/CXCL10/GBP5/IFNG/ncTRIM69 | 0.9206 | 0.9000 | 0.9565 | 0.9554 | 1.8565 |
| CXCL10/GBP5/IFNG/IL19/ncTRIM69 | 0.9206 | 0.9000 | 0.9565 | 0.9424 | 1.8565 |
| CTSS/CXCL10/GBP5/IFNG/IL19/ | 0.9206 | 0.9000 | 0.9565 | 0.9522 | 1.8565 |
| ncTRIM69 | | | | | |
| GBP5/IFNG/ncTRIM69 | 0.9048 | 0.8750 | 0.9565 | 0.9446 | 1.8315 |
| CTSS/GBP5/IFNG/ncTRIM69 | 0.9048 | 0.8750 | 0.9565 | 0.9576 | 1.8315 |
| CTSS/GPB5/IFNG/IL19/ncTRIM69 | 0.9048 | 0.8750 | 0.9565 | 0.9652 | 1.8315 |
| CXCL10/GBP5/IFNG | 0.9048 | 0.9000 | 0.9130 | 0.9522 | 1.8130 |
| CTSS/CXCL10/GBP5/IFNG | 0.9048 | 0.9000 | 0.9130 | 0.9620 | 1.8130 |
| CTSS/CXCL10/GPB5/ncTRIM69 | 0.9048 | 0.9000 | 0.9130 | 0.9478 | 1.8130 |
| CXCL10/GPB5/IFNG/IL19 | 0.9048 | 0.9000 | 0.9130 | 0.9424 | 1.8130 |
| GBP5/IFNG | 0.8889 | 0.8750 | 0.9130 | 0.9533 | 1.7880 |
| CTSS/CXCL10/GBP5 | 0.8889 | 0.8750 | 0.9130 | 0.9500 | 1.7880 |
| CTSS/GPB5/IFNG | 0.8889 | 0.8750 | 0.9130 | 0.9663 | 1.7880 |
| CTSS/GBP5/IFNG/IL19 | 0.8889 | 0.8750 | 0.9130 | 0.9674 | 1.7880 |
| CTSS/CXCL10/GBP5/IFNG/IL19 | 0.8889 | 0.9000 | 0.8696 | 0.9576 | 1.7696 |
| IFNG | 0.8571 | 0.8000 | 0.9565 | 0.9424 | 1.7565 |
| ncTRIM69 | 0.6984 | 0.7250 | 0.6522 | 0.7402 | 1.3772 |

### Example 9: Infection Detection from PBMC Using ncTRIM69-Based Random-Forest Classifier

This example uses the same definitions and abbreviations as defined in Example 6.

The aim of this study was to establish classifiers for preselected ncTRIM69 composing marker combinations enabling a robust identification of individuals infected with tuberculosis pathogens.

In this experiments freshly isolated peripheral blood mononuclear cells (PBMC) of 28 healthy, 28 latently-infected and 30 actively-infected donors (training cohort) were stimulated with ESAT6 and CFP10 antigens as essentially described in example 1 (paragraph “stimulation of PBMCs). In this experiment, patients infected with pathogens causing tuberculosis were preselected with regard to substantial IFNG secretion from isolated PBMC upon stimulation with ESAT6/CFP10 proteins and thus patient collective was biased for the marker IFNG.

RNA isolation was performed as described in example 1. QPCR was performed as described in example 3. Then, random-forest classifiers were established using the software R [3.5.0] in combination with the packages ranger [0.9.0], readxl [1.1.0], stringr [1.3.0] and mlr [2.12.1]. The measurements of the samples described in Table 8 (training samples; N=86, including 28 healthy, 28 latently-infected and 30 actively-infected donors) were log 2-transformed.

Afterwards, the function ranger( ) was used for training with the following parameters: number of trees=1e3, minimal node size=5, split rule=“extratrees” with the number of random splits set to 5 and the number of variables to possibly split at set to 1. The performance of the Random Forest classifier generated on these training samples, for ncTRIM69 alone or in combination with other genes, out of CXCL10, GBP5, IFNG, CTSS and IL19, is shown in Table 12. Established classifiers were independently validated with RNA samples, obtained from specifically stimulated PBMC of an independent set of 56 samples (including 18 healthy, 19 latently-infected and 19 actively-infected donors; see Table 9).

Herein, ncTRIM69 alone had a discriminating power for infection recognition with a sensitivity of 76.3%, a specificity of 88.9% and a score (sensitivity+specificity) of 1.652 (Table 13). The addition of ncTRIM69 to at least 8 combinations of genes, comprising at least one of the following markers: CXCL10, GBP5, IFNG, CTSS and IL19, improved their performance in terms of sensitivity and/or specificity. For instance, the performance of IFNG (sensitivity: 86.8%; specificity: 94.4%; score sensitivity+specificity: 1.813) was improved by ncTRIM69 (IFNG/ncTRIM69; sensitivity: 94.7%; specificity: 94.4%; score sensitivity+specificity: 1.892). Also, the performance of CTSS/IFNG (sensitivity: 89.50%; specificity: 94.4%; score sensitivity+specificity: 1.839) was improved by the addition of ncTRIM69 (CTSS/IFNG/ncTRIM69; sensitivity: 92.1%; specificity: 94.4%; score sensitivity+specificity: 1.865). Similarly, the performance of CXCL10/GBP5/IL19, of CTSS/CXCL10/IL19, of CTSS/CXCL10, of CTSS/CXCL10/IFNG/IL19, of CTSS/CXCL10/GBP5/IFNG, and of CXCL10/IFNG was improved by the addition of ncTRIM69 (Table 13).

Thus, established classifiers for described ncTRIM69 composing marker combinations allow a robust identification of patients infected by tuberculosis pathogens applying samples of freshly isolated PBMC.

TABLE 12
PBMC-based classifier training set (28 non-infected/28 latent TB/30 active TB; N = 86)

| | | | | | Score: |
|---|---|---|---|---|---|
| | | infected.recall | non.infected.recall | | Sum |
| Genes | Accuracy | (sensitivity) | (specificity) | AUC | sens_spec |
| IFNG/ncTRIM69 | 0.9470 | 0.9628 | 0.9144 | 0.9672 | 1.8772 |
| CTSS/CXCL10/IFNG/IL19/ncTRIM69 | 0.9460 | 0.9623 | 0.9129 | 0.9789 | 1.8752 |
| IFNG | 0.9505 | 0.9787 | 0.8915 | 0.9837 | 1.8702 |
| CXCL10/IFNG/IL19/ncTRIM69 | 0.9441 | 0.9638 | 0.9037 | 0.9791 | 1.8676 |
| IFNG/IL19 | 0.9431 | 0.9610 | 0.9061 | 0.9793 | 1.8671 |
| CTSS/CXCL10/IFNG/IL19 | 0.9437 | 0.9628 | 0.9029 | 0.9839 | 1.8657 |
| CTSS/IFNG | 0.9390 | 0.9526 | 0.9124 | 0.9746 | 1.8650 |
| CXCL10/IFNG/IL19 | 0.9413 | 0.9639 | 0.8931 | 0.9831 | 1.8570 |
| CTSS/CXCL10/GPB5/IFNG/IL19 | 0.9398 | 0.9618 | 0.8944 | 0.9836 | 1.8562 |
| IFNG/IL19/ncTRIM69 | 0.9371 | 0.9571 | 0.8968 | 0.9755 | 1.8539 |
| GPB5/IFNG/IL19/ncTRIM69 | 0.9328 | 0.9445 | 0.9089 | 0.9792 | 1.8535 |
| CTSS/GBP5/IFNG/IL19/ncTRIM69 | 0.9320 | 0.9435 | 0.9087 | 0.9774 | 1.8521 |
| GPB5/IFNG/IL19 | 0.9362 | 0.9543 | 0.8976 | 0.9808 | 1.8519 |
| CTSS/IFNG/IL19 | 0.9382 | 0.9611 | 0.8908 | 0.9785 | 1.8519 |
| CXCL10/GBP5/IFNG/IL19/ncTRIM69 | 0.9384 | 0.9605 | 0.8913 | 0.9798 | 1.8518 |
| GPB5/IFNG | 0.9373 | 0.9592 | 0.8913 | 0.9832 | 1.8505 |
| CXCL10/GBP5/IFNG/IL19 | 0.9361 | 0.9560 | 0.8944 | 0.9830 | 1.8504 |
| CTSS/CXCL10/IL19 | 0.9360 | 0.9577 | 0.8916 | 0.9811 | 1.8493 |
| CXCL10/IFNG/ncTRIM69 | 0.9367 | 0.9587 | 0.8905 | 0.9761 | 1.8493 |
| CXCL10/IFNG | 0.9363 | 0.9617 | 0.8841 | 0.9802 | 1.8458 |
| CTSS/CXCL10/GPB5/IFNG/IL19/ncTRIM69 | 0.9333 | 0.9543 | 0.8896 | 0.9810 | 1.8439 |
| CXCL10/IL19 | 0.9351 | 0.9602 | 0.8837 | 0.9806 | 1.8439 |
| CTSS/GPB5/IFNG/IL19 | 0.9323 | 0.9506 | 0.8933 | 0.9808 | 1.8439 |
| CTSS/GBP5/IFNG | 0.9319 | 0.9518 | 0.8896 | 0.9790 | 1.8414 |
| GPB5/IFNG/ncTRIM69 | 0.9299 | 0.9485 | 0.8911 | 0.9787 | 1.8396 |
| CXCL10/GBP5/IFNG/ncTRIM69 | 0.9298 | 0.9496 | 0.8889 | 0.9779 | 1.8385 |
| CTSS/CXCL10/IFNG | 0.9311 | 0.9524 | 0.8853 | 0.9807 | 1.8378 |
| CTSS/CXCL10/IFNG/ncTRIM69 | 0.9280 | 0.9458 | 0.8907 | 0.9789 | 1.8365 |
| CXCLIO/GBP5/IFNG | 0.9285 | 0.9487 | 0.8864 | 0.9817 | 1.8351 |
| CXCL10/IL19/ncTRIM69 | 0.9307 | 0.9589 | 0.8736 | 0.9783 | 1.8325 |
| CTSS/GBP5/IFNG/ncTRIM69 | 0.9254 | 0.9437 | 0.8871 | 0.9759 | 1.8308 |
| CTSS/CXCL10/IL19/ncTRIM69 | 0.9267 | 0.9496 | 0.8811 | 0.9763 | 1.8307 |
| CTSS/CXCL10/GBP5/IFNG | 0.9258 | 0.9474 | 0.8807 | 0.9798 | 1.8280 |
| CTSS/IFNG/ncTRIM69 | 0.9201 | 0.9357 | 0.8901 | 0.9674 | 1.8259 |
| CXCL10/GPB5/IL19 | 0.9253 | 0.9496 | 0.8761 | 0.9812 | 1.8258 |
| CTSS/IFNG/IL19/ncTRIM69 | 0.9233 | 0.9458 | 0.8781 | 0.9723 | 1.8240 |
| CTSS/CXCL10/GPB5/IFNG/ncTRIM69 | 0.9204 | 0.9387 | 0.8819 | 0.9797 | 1.8206 |
| GPB5/IL19/ncTRIM69 | 0.9151 | 0.9312 | 0.8841 | 0.9720 | 1.8153 |
| CTSS/CXCL10/GPB5/IL19 | 0.9210 | 0.9482 | 0.8640 | 0.9816 | 1.8122 |
| GPB5/LL19 | 0.9130 | 0.9335 | 0.8716 | 0.9743 | 1.8051 |
| CTSS/GBP5/IL19/ncTRIM69 | 0.9113 | 0.9310 | 0.8735 | 0.9707 | 1.8045 |
| CXCL10/GBP5/IL19/ncTRIM69 | 0.9189 | 0.9508 | 0.8529 | 0.9794 | 1.8037 |
| CTSS/GPB5/IL19 | 0.9099 | 0.9371 | 0.8544 | 0.9750 | 1.7915 |
| CTSS/CXCL10/GBP5/IL19/ncTRIM69 | 0.9086 | 0.9420 | 0.8405 | 0.9779 | 1.7825 |
| CTSS/CXCL10/GBP5 | 0.8898 | 0.9236 | 0.8209 | 0.9752 | 1.7445 |
| CTSS/GPB5 | 0.8871 | 0.9175 | 0.8239 | 0.9697 | 1.7414 |
| CXCL10/GBP5/ncTRIM69 | 0.8875 | 0.9265 | 0.8084 | 0.9714 | 1.7349 |
| CTSS/CXCL10 | 0.8837 | 0.9152 | 0.8188 | 0.9723 | 1.7340 |
| CXCL10/GBP5 | 0.8884 | 0.9296 | 0.8035 | 0.9724 | 1.7330 |
| GBP5 | 0.8848 | 0.9212 | 0.8104 | 0.9723 | 1.7316 |
| CTSS/GBP5/ncTRIM69 | 0.8792 | 0.9105 | 0.8156 | 0.9633 | 1.7261 |
| CTSS/CXCL10/ncTRIM69 | 0.8794 | 0.9150 | 0.8095 | 0.9687 | 1.7244 |
| GBP5/ncTRIM69 | 0.8794 | 0.9148 | 0.8064 | 0.9630 | 1.7212 |
| CTSS/CXCL10/GBP5/ncTRIM69 | 0.8806 | 0.9196 | 0.8011 | 0.9743 | 1.7207 |
| CXCL10/ncTRIM69 | 0.8788 | 0.9170 | 0.8017 | 0.9625 | 1.7187 |
| CXCL10 | 0.8673 | 0.8995 | 0.7997 | 0.9682 | 1.6992 |
| CTSS/IL19/ncTRM69 | 0.8583 | 0.8997 | 0.7753 | 0.9371 | 1.6750 |
| CTSS/ncTRIM69 | 0.8424 | 0.8649 | 0.7969 | 0.9157 | 1.6618 |
| IL19/ncTRIM69 | 0.8520 | 0.9047 | 0.7437 | 0.9340 | 1.6484 |
| ncTRIM69 | 0.8348 | 0.8670 | 0.7691 | 0.8767 | 1.6361 |

TABLE 13
PBMC-based classifier test set (18 non-infected/19 latent TB/19 active TB; N = 56)

| | | | | | score: |
|---|---|---|---|---|---|
| | | infected.recall | noninfected.recall | | sum |
| Genes | Accuracy | (sensitivity) | (specificity) | AUC | sens + spec |
| IFNG/ncTRIM69 | 0.946 | 0.947 | 0.944 | 0.963 | 1.892 |
| CXCL10/IFNG/ncTRIM69 | 0.946 | 0.947 | 0.944 | 0.961 | 1.892 |
| CXCL10/IFNG | 0.929 | 0.921 | 0.944 | 0.976 | 1.865 |
| CTSS/CXCL10/IFNG/IL19/ncTRIM69 | 0.929 | 0.921 | 0.944 | 0.962 | 1.865 |
| CTSS/IFNG/ncTRIM69 | 0.929 | 0.921 | 0.944 | 0.953 | 1.865 |
| CTSS/CXCL10/GBP5/IFNG/ncTRIM69 | 0.929 | 0.921 | 0.944 | 0.950 | 1.865 |
| CTSS/IFNG | 0.911 | 0.895 | 0.944 | 0.963 | 1.839 |
| CTSS/CXCL10/GBP5/IFNG | 0.911 | 0.895 | 0.944 | 0.962 | 1.839 |
| IPNG | 0.893 | 0.868 | 0.944 | 0.969 | 1.813 |
| CTSS/CXCL10/ncTRIM69 | 0.875 | 0.868 | 0.889 | 0.934 | 1.757 |
| CTSS/CXCL10/IFNG/IL19 | 0.875 | 0.842 | 0.944 | 0.968 | 1.787 |
| CXCL10/GBP5/IL19/ncTRIM69 | 0.875 | 0.842 | 0.944 | 0.959 | 1.787 |
| CTSS/CXCL10/IL19/ncTRIM69 | 0.839 | 0.816 | 0.889 | 0.944 | 1.705 |
| CTSS/CXCL10 | 0.839 | 0.816 | 0.889 | 0.944 | 1.705 |
| CTSS/CXCL10/IL19 | 0.857 | 0.789 | 1.000 | 0.952 | 1.789 |
| CXCL10/GBP5/IL19 | 0.839 | 0.789 | 0.944 | 0.963 | 1.734 |
| ncTRIM69 | 0.804 | 0.763 | 0.889 | 0.855 | 1.652 |

### Example 10: Infection Detection in Actively with Mtb Infected Patients Under Treatment with Rifampicin

Detection of infection with Mtb also works in actively infected patients under initiation of antibacterial therapy. Rifampicin is an often utilized antibiotic to initiate treatment of TB.

To test the influence of rifampicin on the detectability of Mtb infection three patients with active TB were tested with the method described here before initiation of therapy (day 0) and after approximately one week rifampicin therapy (day 6 till day 10). An active donor without rifampicin treatment served as control.

For this purpose blood was drawn from patients with active TB (ATB) at the two consecutive time points each. Whole blood samples were then stimulated with CFP10 and ESAT6, and RNA was isolated as described in example 1. The isolated RNA was used for cDNA synthesis and qPCR analysis as described in example 3. For all stimulated or unstimulated samples qPCRs on marker-genes IFNG, CXCL10, GBP5, and ncTRIM69, as well as on the housekeeping gene RPLP0 were performed.

RPLP0 was used to normalize marker-gene expression and differences between stimulated and non-stimulated samples from one donor was used to calculate the fold change as described in example 4.

Finally the patient's infection state utilizing the fold change values for the markers was evaluated for IFNG alone as reference or in combinations via a random forest derived classifier (examples 6) indicating a probability of being infected. Donor 3 would have been classified incorrectly after 10 days of rifampicin treatment if only IFNG would have been considered. The addition of information of GBP5, ncTRIM69 or CXCL10 fold change values leads to a correct classification of this donor (FIG. 1).

In all other cases the classification by the different classifiers were concordant.

1. An in vitro method of detecting an infection with pathogens causing tuberculosis comprising the steps:
a) contacting a first aliquot of a sample of an individual with at least one antigen of a pathogen causing tuberculosis,
b) incubating the first aliquot with the at least one antigen over a certain period of time,
c) detecting in the first aliquot and in a second aliquot of the sample of the individual at least two markers using reverse transcription quantitative real-time polymerase chain reaction (RT-qPCR) or RNA Sequencing (RNA-Seq), wherein the second aliquod has not been incubated with the at least one antigen, and wherein one of the at least two markers is IFN-γ or CXCL10 and the other of the at least two markers is either a distinct one of IFN-γ, or CXCL10 or one of ncTRIM69, GBP5, CTSS and IL19, and
d) comparing the detected markers in the first aliquot with the detected markers in the second aliquot.

2. The in vitro method according to claim 1, wherein in step c) one of the at least two markers is IFN-γ or CXCL10 and the other of the at least two markers is one of ncTRIM69, GBP5, CTSS and IL19.

3. The in vitro method according to claim 1, wherein in step c) a marker combination is detected comprising or consisting of one of the following combinations:
IFN-γ and GBP5
IFN-γ and ncTRIM69
IFN-γ and CTSS
IFN-γ and IL19
CXCL10 and GBP5
CXCL10 and ncTRIM69
CXCL10 and CTSS
CXCL10 and IL19

4. The in vitro method according to claim 1, wherein at least a third, optionally a fourth, optionally a fifth and optionally a sixth marker is detected wherein the at least third, fourth, fifth or sixth marker is selected from the group consisting of: IFN-γ, CXCL10, GBP5, ncTRIM69, CTSS and IL19, with the provision that the first, second, third and optionally fourth, fifth and sixth marker are each distinct markers.

5. The in vitro method according to claim 1, wherein at least a third marker is detected, wherein two of the at least three markers are IFN-γ, CXCL10 or GBP5 and the other of the at least three markers is either a distinct one of IFN-γ, CXCL10, or GBP5 or one of ncTRIM69, CTSS and IL19.

6. The in vitro method according to claim 1, wherein in step c) a marker combination is detected comprising or consisting of one of the following combinations:
IFN-γ, GBP5, and CXCL10
IFN-γ, GBP5, CXCL10, and ncTRIM69
CXCL10, GBP5, IFN-γ, and CTSS
IFN-γ, CXCL10, and CTSS
CTSS, CXCL10, GBP5, IFN-γ, and ncTRIM69
CXCL10, IFN-γ, and ncTRIM69
CXCL10, IFN-γ, and IL19
CXCL10, IFN-γ, IL19, and ncTRIM69
CTSS, CXCL10, IFN-γ, and ncTRIM69
CTSS, CXCL10, IFN-γ, IL19, and ncTRIM69
GBP5, IFN-γ, and ncTRIM69
CTSS, GBP5, and IFN-γ
IFN-γ, GBP5, CXCL10, IL19, and ncTRIM69
CXCL10, IFN-γ, IL19, and GBP5
CXCL10, GBP5, and ncTRIM69
CTSS, CXCL10, IFN-γ, and IL19
CTSS, CXCL10, GBP5, IFN-γ, and IL19
CTSS, CXCL10, GBP5, IFN-γ, IL19, and ncTRIM69
CTSS, CXCL10, GBP5, and ncTRIM69
CXCL10, GBP5, IL19, and ncTRIM69
CTSS, CXCL10, and GBP5
CTSS, GBP5, IFN-γ, and ncTRIM69
GBP5, IFN-γ, IL19, and ncTRIM69
CTSS, GBP5, IFN-γ, IL19, and ncTRIM69
CTSS, CXCL10, GBP5, IL19, and ncTRIM69
IFN-γ, GBP5, IL-19

7. The in vitro method according to claim 1, wherein in step c) a marker combination is detected comprising or consisting of the combination IFN-γ and CXCL10.

8. The in vitro method according to claim 1, wherein in step c) a marker combination is detected comprising or consisting of one of the following combinations:
CXCL10, IL19, and ncTRIM69
CTSS, IFN-γ, ncTRIM69
CTSS, IFN-γ, IL19, and ncTRIM69
CTSS, CXCL10, and ncTRIM69
IFN-γ, IL19, and ncTRIM69
CTSS, CXCL10, IL19, and ncTRIM69

9. An in vitro method of detecting an infection with pathogens causing tuberculosis comprising the steps:
(a) contacting a first aliquot of a sample of an individual with at least one antigen of a pathogen causing tuberculosis,
b) incubating the first aliquot with the at least one antigen over a certain period of time,
c) detecting in the first aliquot and in a second aliquot of the sample of the individual at least one marker using quantitative PCR (qPCR), reverse transcription quantitative real-time polymerase chain reaction (RT-qPCR), RNA Sequencing (RNA-Seq), expression profiling and microarray, wherein the second aliquod has not been incubated with the at least one antigen, and wherein the at least one marker is ncTRIM69, and
d) comparing the detected marker(s) in the first aliquot with the detected marker(s) in the second aliquot.

10. The in vitro method according to claim 9, wherein in step c) at least a second marker is detected in the first aliquot and in the second aliquot, wherein the second marker is selected from the group consisting of: IFN-γ, CXCL10, GBP5, CTSS and IL19, in particular, wherein in step c) a marker combination is detected comprising or consisting of one of the following combinations:
IL19, and ncTRIM69
IFN-γ, and ncTRIM69
IFN-γ, IL19, and ncTRIM69
IFN-γ, IL19, and ncTRIM69
GBP5, and ncTRIM69
GBP5, IL19, and ncTRIM69
GBP5, IFN-γ, and ncTRIM69
GBP5, IFN-γ, IL19, and ncTRIM69
CXCL10, and ncTRIM69
CXCL10, IL19, and ncTRIM69
CXCL10, IFN-γ, and ncTRIM69
CXCL10, IFN-γ, IL19, and ncTRIM69
CXCL10, GBP5, and ncTRIM69
CXCL10, GBP5, IL19, and ncTRIM69
CXCL10, GBP5, IFN-γ, and ncTRIM69
CXCL10, GBP5, IFN-γ, IL19, and ncTRIM69
CTSS, and ncTRIM69
CTSS, IL19, and ncTRIM69
CTSS, IFN-γ, and ncTRIM69
CTSS, IFN-γ, IL19, and ncTRIM69
CTSS, GBP5, and ncTRIM69
CTSS, GBP5, IL19, and ncTRIM69
CTSS, GBP5, IFN-γ, and ncTRIM69
CTSS, GBP5, IFN-γ, IL19, and ncTRIM69
CTSS, CXCL10, and ncTRIM69
CTSS, CXCL10, IL19, and ncTRIM69
CTSS, CXCL10, IFN-γ, and ncTRIM69
CTSS, CXCL10, IFN-γ, IL19, and ncTRIM69
CTSS, CXCL10, GBP5, and ncTRIM69
CTSS, CXCL10, GBP5, IL19, and ncTRIM69
CTSS, CXCL10, GBP5, IFN-γ, and ncTRIM69
CTSS, CXCL10, GBP5, IFN-γ, IL19, and ncTRIM69

11. The in vitro method according to claim 1, wherein the sample is or comprises a body fluid, in particular blood, more particularly whole blood or anticoagulated whole blood, lymph, a bronchial lavage, or a suspension of lymphatic tissue or comprises isolated cells from said body fluids, in particular a purified or isolated PBMC population, or isolated cells of a bronchial lavage.

12. The in vitro method according to claim 1, wherein the at least one antigen of a pathogen causing tuberculosis is a peptide, oligopeptide, a polypeptide, a protein, a RNA or a DNA.

13. The in vitro method according to claim 1, wherein step (a) comprises contacting a first aliquot of a sample of an individual with two, three, four, five, six, seven, eight, nine, ten or more antigens of a pathogen causing tuberculosis, in particular wherein said antigens are selected from the group consisting RD-1 antigens, ESAT-6, CFP10, TB7.7, Ag 85, HSP-65, Ag85A, Ag85B, MPT51, MPT64, TB10.4, Mtb8.4, hspX, Mtb12, Mtb9.9, Mtb32A, PstS-1, PstS-2, PstS-3, MPT63, Mtb39, Mtb41, MPT83, 71-kDa, PPE68 and LppX, H1-hybrid, AlaDH, Ag85B, Pst1S, Ag85, ORF-14, Rv0134, Rv0222, Rv0934, Rv1256c, Rv1514c, Rv1507c, Rv1508c, Rv1511, Rv1512, Rv1516c Rv1766 Rv1769 Rv1771, Rv1860, Rv1974 Rv1976c Rv1977, Rv1980c, Rv1982c, Rv1984c, Rv1985c, Rv2031c, Rv2074, Rv2780, Rv2873 Rv3019c, Rv3120, Rv3615c Rv3763, Rv3871, Rv3872, Rv3873, Rv3876, Rv3878, Rv3879c, Rv3804c, Rv3873, Rv3878, Rv3879c, Rv3879c, Rv1508c, Rv3876, Rv1979c, Rv2655c, Rv1582c, Rv1586c, Rv3877, Rv2650c, R1576c, Rv1256c, Rv3618, Rv2659, cRv1770, Rv1771, Rv1769, Rv3428c, Rv1515c, Rv1511, Rv1512, Rv1977, Rv1985c, Rv0134, Rv1509, Rv3427c, Rv2646, Rv1041, cRv1507c, Rv1980c, Rv1514c, Rv1190, Rv3878, Rv1969, Rv1975, Rv1968, Rv1971, Rv3873, Rv2652c, Rv2651c, Rv1585c, Rv1577c, Rv1972, Rv1507A, Rv1506c, Rv1966, Rv1973, Rv1573, Rv1578c, Rv1974, Rv1575, Rv2645, Rv1987, Rv1970, Rv2074, Rv1976c, Rv2073c, Rv2810c, Rv1581c, Rv3136A, Rv2548A, Rv3098A, Rv2231A, Rv2647, Rv1772, Rv1508A, Rv2658c, Rv1767, Rv2063A, Rv1954, ARv1583c, Rv2656c, Rv0724A, Rv3875, Rv2348c, Rv0222, Rv2653c, Rv1580c, Rv1579c, Rv1766, Rv1366A, Rv3874, Rv0061c, Rv1768, Rv0397A, Rv1991A, Rv2274A, Rv3617, Rv1574, Rv3350c, Rv1984c, Rv2801A, Rv3872, Rv2657c, Rv1983, Rv2142A, Rv1967, Rv2862A, Rv3190A, Rv2237A, Rv2468A, Rv1982A, Rv1982c, Rv1584c, Rv0691A, Rv2395A, Rv2654c, Rv2231B, Rv1257c, Rv2395B, Rv1516c, Rv0186A, Rv0530A, Rv0456B, Rv3120, Rv3738c, Rv3121, Rv3426, Rv3621c, Rv0157A, Rv2349c, Rv1965, Rv3508, Rv3514, Rv0500B, Rv1978, Rv2350c, Rv2351c, Rv1986, Rv3599c, Rv2352c, Rv1255c, Rv2356c, Rv2944, and Rv3507 or a polypeptide mixture, such as tuberculin PPD.

14. The in vitro method according to claim 1, wherein step (a) comprises contacting a first aliquot of a sample of an individual with at least two antigens, in particular with CFP10 and ESAT6.

15. The in vitro method according to claim 1, wherein step d) is performed by analysing a detectable change in marker expression in the first aliquod in comparison to the second aliquod, preferably above a certain threshold, preferably by a classification method, by fold change analysis, and/or by analyzing a change of the absolute amount of marker mRNA in the first and the second aliquod, in particular wherein the classification method is at least one of artificial neural networks, logistic regression, decision trees, Random Forest, Least Absolute Shrinkage and Selection Operator (LASSO), support vector machines (SVMs), threshold analysis, linear discriminant analysis, k-Nearest Neighbor (kNN), Naive Bayes and Bayesian Network.

16. The in vitro method according to claim 1, wherein a difference in marker expression in the first and second aliquot is indicative that the individual is infected with pathogens causing tuberculosis or has been in contact with pathogens causing tuberculosis.

17. The in vitro method according to claim 1, wherein the marker ncTRIM69 is encoded by a nucleic acid molecule comprising a nucleic acid sequence according to SEQ ID NO: 9, 10 or 11 or a functional variant thereof having at least 70%, 75%, 80%, 85%, 90% or 95% sequence identity to a sequence according to SEQ ID NO: 9, 10 or 11.

18. A kit comprising at least one antigen, and
(i) at least two primer pairs for amplification of the at least two markers which are detected in step c) of claim 1, and preferably at least two probes for detecting the at least two markers, and/or
(ii) at least one primer pair for amplification of the marker ncTRIM69, wherein the primer pair comprises preferably nucleic acid sequences according to SEQ ID NO: 12 and 13 or nucleic acid sequences according to SEQ ID NO: 14 and 15, and preferably at least one probes for detecting the marker ncTRIM69, wherein the probe comprises preferably a nucleic acid sequence according to SEQ ID NO: 16 or 17, optionally linked to a fluorescence dye and/or a quencher.

19. An in vitro method of detecting infection with pathogens causing tuberculosis comprising detecting marker ncTRIM69, which is encoded by a nucleic acid molecule comprising a nucleic acid sequence according to SEQ ID NO: 9, 10 or 11 or a functional variant thereof having at least 70%, 75%, 80%, 85%, 90% or 95% sequence identity to a nucleic acid sequence according to SEQ ID NO: 9, 10 or 11.
